(19) United States
(12) Patent Application Publication
(10) Pub. No.: US 2016/0347829 A1
(43) Pub. Date: Dec. 1, 2016

(54) Compositions containing antimicrobial IgY antibodies, for treatment and prevention of disorders and diseases caused by oral health compromising (OHC) microorganisms

(71) Applicant: MERIAL INC., DULUTH, GA (US)

(72) Inventors: Robert Nordgren, Watkinsville, GA (US); Monica Dias Figueiredo, Athens, GA (US)

(73) Assignee: MERIAL INC., DULUTH, GA (US)

(21) Appl. No.: 15/162,472

(22) Filed: May 23, 2016

Related U.S. Application Data
(60) Provisional application No. 62/167,082, filed on May 27, 2015.

(51) Int. Cl. C07K 16/12; A61Q 11/00; A61K 8/04; A61K 8/64; A61K 8/98

(52) U.S. Cl. CPC C07K 16/1275; C07K 16/1203; A61K 8/64; A61K 8/982; A61K 8/042; A61Q 11/00; C07K 2317/23; C07K 2317/11; A61K 2800/74

(57) ABSTRACT

The present invention encompasses methods and compositions for inhibiting, treating, and preventing dental diseases in human and non-human animals, particularly domesticated companion animals.

### CROSS-REFERENCE TO RELATED APPLICATIONS

This application claims priority to provisional application U.S. Ser. No. 62/167,082, filed on 27 May 2016, and herein incorporated by reference in its entirety.

### INCORPORATION BY REFERENCE

Any foregoing applications and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. Citation or identification of any such document in this application is not an admission that such document is available as prior art to the present invention and does not reflect any view of the validity, patentability and/or enforceability of such cited patent documents. All sequences referenced herein by GenBank Accession numbers are herein incorporated by reference in their entirety, and said sequences are as set forth in GenBank at as of the filing date of the present application.

### FIELD OF THE INVENTION

The present invention encompasses inoculation of avians for the production of antimicrobial, including anti-adhesive, IgY antibodies, which are useful in preventing and reducing dental carries and improving oral health. The present invention further relates to oral hygiene and specifically to methods of treating the oral cavity with a dental delivery systems such as toothpaste, masticables including chews, dental floss or toothpicks, with improved cleaning, conditioning and antimicrobial properties, which provide the teeth with a protective barrier against oral health compromising (OHC) microorganisms. The present invention also relates to compositions and dental delivery systems having improved cleaning, conditioning, and antimicrobial properties, which provide the teeth with an impervious protective barrier.

### SUMMARY OF THE INVENTION

Tooth and gum disease can lead to serious health problems both in humans and companion animals. However, dogs and cats make use of their teeth more than humans do. Accordingly, toothache, dental disease, and loss of teeth can all have serious consequences for companion animals. To date, damage to the teeth and gums in companion animals is considered to be permanent and irreversible. According to the American Veterinary Dental Society, 80% of dogs and 70% of cats have periodontal (gum) disease by the age of three. Proper dental care could increase the life of these animals by many years. Accordingly, maintenance of good oral health and prevention of oral disease is a primary necessity for animals, which, unlike humans, do not have the ability to exercise control over oral and dental hygiene by using proper preventative techniques.

Dental care in dogs and cats has become common. Like humans, dog teeth and gums are also susceptible to many of the same oral health problems (e.g., gingivitis and periodontal disease). However, unlike humans, animals rarely get cavities. This is because cavities are primarily caused by the high sugar content of the human diet and the morphology of human teeth. However, periodontal disease affects both human and mammals alike. Bacteria and plaque that attaches to the tissues of the mouth can cause periodontal disease. The first stage of periodontal disease is gingivitis, which is very common. In this stage, the bacteria have mixed with saliva and formed plaque. Plaque adheres to the teeth and hardens, forming tartar and calculus. These tartar deposits irritate the gum tissue and cause inflammation, swelling, and infection. It is at this stage that gingivitis is most notable.

There are indications that oral health status has a profound effect on an animal's general health. Periodontal disease and other oral cavity pathologies may cause bacteria and/or their toxins to enter the bloodstream with potentially deleterious effects on various internal organs (e.g., the heart, kidneys, liver, etc.). Conversely, poor systemic health may manifest in the oral cavity in various ways and may also exacerbate periodontal disease. An animal's dental examination is therefore not always limited to the oral cavity, but frequently includes a general physical examination. Laboratory examinations, to evaluate systemic disease concerns, are also commonly performed. Some dogs and cats suffer from chronic oral infection (e.g., stomatitis, a poorly understood condition that is difficult to treat) and oral cancer.

Moreover, approximately 80% of dogs and cats demonstrate some degree of periodontal disease by four years of age (Harvey C E and Emily P P., 1993). The progression and etiology of periodontal disease in companion animals is similar to that of human periodontitis (Hennet et al., 1992 J Vet Dent). Porphyromonas-like black pigmented anaerobic bacteria (BPAB) have been identified in dog periodontal pockets (Harvey et al., 1995 J Vet Dent). Relevant species include, but are not limited to, Porphyromonas gulae, Porphyromonas salivosa, Porphyromonas denticanis, and are able to induce bone loss in a murine model of periodontitis (Hardham et al., 2005 Vet Microbiol).

In addition to small molecule antibiotic strategies, some companies have explored the possibility of vaccinating animals to protect them against the bacteria that cause oral health problems, including dental caries and periodontitis. For example, Pfizer obtained conditional approval for a three-valent bacterin vaccine containing inactivated P. gulae, P. salivosa and P. denticanis. However, approval was withdrawn owing to lack of efficacy.

In a related example, passive oral administration of IgY (known as hyperimmune γ-livetin from egg yolk) antibody against S. mutans GBP-B, in the context of a rat model of dental caries, has shown promise (Smith et al., Infect Immun. 2001 May; 69(5): 3135-3142). Streptococcus mutans are gram-positive cocci shaped bacteria. These facultative anaerobes are commonly found in the oral cavity, and are a major contributor of tooth decay. Streptococcus mutans is a cariogenic microorganism that breaks down sugar for energy and produces an acidic environment, which demineralizes the superficial structure of the tooth. The result of the conversion disintegrates the coating of the tooth then later dissolves the Calcium molecule creating a hole.

Another group has achieved some success using a lozenge to deliver anti-cell-associated glucosyltransferase immunoglobulin Y salivary mutans streptococci in healthy young adults to reduce adherence of S. mutans to teeth (Nguyen et al., J Am Dent Assoc 2011 August; 142(8): 943-9).

Recently, one group has demonstrated that the P. gulae strains, which produce “gingipain-like” enzymes, are susceptible to passive immunization by anti-gingipain IgY. (Rahman A. K. M. Shofiqur et al., (2011) Vet Sci Dev). Further, anti-gingipain antibody inhibited alveolar bone resorption induced by P. gulae. (K. Watanabe et al. 2013 IADR Seattle).

The growing interest in IgY technology stems from the numerous advantages it offers compared with using its mammalian equivalent, IgG. The primary advantage of obtaining Ig via laying hens instead of mammals is improved animal welfare. It is a refinement of the antibody production protocol because it does not involve bleeding the antibody producer animals, unlike the mammals models, and the yield is significantly higher. Moreover, the long-lasting titers obtained from laying hens also reduce the need for frequent booster injections (Schade et al., 2005). In addition, there are also numerous efficient and inexpensive IgY extraction processes (De Meulenaer et al., 2001), and the highly specific, hyper-immune yolk may even be used as is.

Sirsat et al. (2009) noted some risk in developing overly specific tools, but this risk is minimal in the case of polyclonal egg yolk antibodies. When Chalghoumi et al. (2008) developed IgY specific to two Salmonella serovars, they demonstrated a high level of cross-reactivity of IgY developed against a particular serovar with antigens of the other one, and vice versa. Thus, using vaccine antigens shared among several serovars addresses the risk of the developed IgY being too highly specific. In addition, the fact that Chalghoumi et al. (2008) were able to raise IgY against two Salmonella serovars in a single egg yolk indicates that it could soon be possible to develop real “cocktail eggs” targeting a diverse set of organisms. Finally, the use of IgY does not lead to undesirable side effects, disease resistance or toxic residues (Xu et al., 2011), unlike other drug strategies (e.g., antibiotics).

The standard protocol for producing antigen-specific IgY intended for passive dietary immunization in animals is known in the art. Hens are usually exposed to the targeted antigen through an injection. This triggers a humoral immune response that manifests itself initially by the production of specific IgY in the blood serum of the immunized hen, followed by its export in the yolk of laid eggs. Once the immune response has been induced, the transovarial passage of IgY takes about 6-7 days (Bollen et al., 1997). The composition of the pool of IgY in the yolk is clearly related to that in the hens' circulating blood (Hamal et al., 2006). Immunoglobulin Y (IgY), previously referred to as “egg yolk antibodies,” represents the functional equivalent of the mammalian IgG in birds, reptiles, and amphibians. In birds, IgY is continually synthesized and secreted into the bloodstream and transferred into the egg yolk (FIG. 1), where it accumulates in large quantities (60 to 150 mg per egg yolk) and at high titers, for long periods of time. The role of egg-yolk antibodies is to provide a passive antibody source to offspring against common avian pathogens until full maturation of their own immune system. IgY has a short half-life (˜36 h), which is considerably shorter than the half-life of mammalian IgG, and this feature is suggested to help avoid immune recognition (As reviewed in Suartini 2014†). As an immuno-intervention strategy, IgY presents several advantages over mammalian IgG: 1) IgY exhibits up to five times higher affinity and reactivity to a specific antigen than IgG when tested in competition assays (Rahman, 2014), 2) IgY does not bind or activate mammalian complement or Fc receptors, does not bind protein A,G, or rheumatoid factor and therefore cannot elicit non-specific inflammatory responses, particularly in the gastrointestinal tract, when administered orally (Rahman, 2014). Furthermore, IgY does not interfere with mammalian IgG in serological tests.

The first step in specific IgY production is to choose the target antigen. This can be a single antigen (protein, peptides or polysaccharides) or a complex multi-antigen (bacteria, mold, viruses or parts of these) (FIG. 2). The molecules exhibiting the best immunogenicity are proteins (Schwarzkopf et al., 2001). In the case of small antigens with a molecular weight below 12,000 da (known as “haptens”), conjugation to a carrier protein (e.g., bovine gamma globulin) is often required (Cook et al., 2010). Carbohydrates and nucleic acids could also be coupled advantageously with carriers because of their reduced immunogenicity (Schwarzkopf et al., 2001). Apart from the intrinsic immunogenicity of the target antigen, its quality and quantity should also be taken into account. The purity of the antigen is a crucial parameter because impurities could lead to IgY with more activity against the impurities themselves than against the antigen of interest (Leenaars et al., 2005). In addition, contaminations of the antigen with microbes, endotoxins or chemical residues from the inactivation/extraction process could have a negative effect on animal welfare as well as on immune response (Leenaars et al., 2005). The antigen dose is also critical because too much or too little antigen can lead to suppression, sensitization, tolerance or other undesirable immunomodulatory effects (Schwarzkopf et al., 2001). The recommended amount of a soluble protein to be administered in a given vaccine dose is usually in the range of 0.01 mg to 1 mg (Schwarzkopf et al., 2001; Cook et al., 2010).

However, until the instant disclosure, no effective IgY-based therapeutic compositions for treating, reducing and preventing oral health problems, including dental caries, were known.

Accordingly, Applicants set out to provide effective IgY-based compositions and methods of making an use thereof, to assist in the maintenance of good oral health as well as the prevention and treatment of oral disease in companion animals.

The present disclosure provides novel IgY antibody compositions and methods of making and use thereof. IgY antibodies may include those produced using gingipain polypeptides, including chimeric gingipain polypeptides. Some particularly useful polypeptides are disclosed in US 2003/0232022 A1 (to CSL Limited), the disclosure of which is herein incorporated by reference in its entirety. Related polypeptides, including related chimeric polypeptides, are expected to produced comparable results.

In an embodiment, obtaining specific IgY involves injecting an antigen-adjuvant combination at certain intervals. Numerous protocols, using different antigens, adjuvants, injection routes and intervals between injections, have been described over the years. All these factors are critical because they influence both the outcome of the immunization procedure (amount and specificity of the obtained IgY) and the welfare of the hens.

The following is a non-exhaustive list of representative microorganisms, portions thereof, and proteins thereof, which may be used to produce the IgY and IgY-containing compositions of the instant disclosure: streptococci, lactobacilli, staphylococci, corynebacteria, and various anaerobes in particular bacteroidetes, including Porphyromonas gingivalis. P. gingivalis is found in the oral cavity, where it is implicated in certain forms of periodontal disease, as well as in the upper GI tract, the respiratory tract, and the colon.

In particular embodiments, the microorganism may include Streptococcus salivarius, Streptococcus mutans, Streptococcus sanguinis and Streptococcus sobrinus. The family of adhesions from S. mutans and S. sobrinus has been shown to be effective antigens, and so Applicants envision that any of said antigens could be used to elicit production of specific IgY antibodies to block microorganisms that negatively impact oral-health.

In other embodiments, the microorganism may include Fusospirochetes, Veillonella, Actinobacillus actinomycetemcomitans,

Accordingly, it is a primary object of this disclosure to provide compositions and methods for blocking or interfering with the adherence of the foregoing microorganisms from the teeth and oral cavity of animals, including dogs and cats.

In an embodiment, the disclosure provides an immunogenic component that is used to elicit production in chicken specific oral disease blocking (ODB) IgY antibodies.

In some embodiments, the immunogenic component may include the following, or substantially immunogenically equivalent derivatives, fragments, or portions thereof: GenBank accession numbers having significant identity to S. salivarius, S. mutans, S. sanguinis and S. sobrinus oral health-relevant polypeptides, including, the surface fibrillar adhesions known as AgI/II, the glucosyltransferases (GTF) and the glucan-binding proteins (GBP).

In a particular embodiment, the immunogenic component may be an N-terminal fragment of AgI/II protein from Streptococcus mutans GS-5. The component may be the surface adhesin protein identified in GenBank as AFM81671.1 (gi392603507).

The immunogenic component may have significant identity to AgI/II, and may include homologous portions of the sequences represented by the following GenBank accession numbers, or sequences having at least 90, 91, 92, 93, 94, 95, 96, 97, 98 or 99% or greater, identity thereto: gi488214056, gi488225222, gi518152415, gi488227403, gi488279945, gi518147219, gi488223981, gi488203641.

In particular embodiments, the immunogenic component may comprise, consist of, or consist essentially of one or more of the following, or substantially immunogenically equivalent derivatives, fragments, or portions thereof, or sequences having at least 90, 91, 92, 93, 94, 95, 96, 97, 98 or 99% or greater, identity thereto: GenBank accession numbers gi1066835 (SEQ ID NO:227), gi1246379 (SEQ ID NO:228), gi167729944 (SEQ ID NO:229), gi167730167 (SEQ ID NO:230), gi1813996 (SEQ ID NO:231), gi188595513 (SEQ ID NO:232), gi1890077 (SEQ ID NO:233), gi2182812 (SEQ ID NO:234), gi2738803 (SEQ ID NO:235), gi2827775 (SEQ ID NO:236), gi298274962 (SEQ ID NO:237), gi333803263 (SEQ ID NO:238), gi363646003 (SEQ ID NO:239), gi376316122 (SEQ ID NO:240), gi392611916 (SEQ ID NO:241), gi4103639 (SEQ ID NO:242), gi45593253 (SEQ ID NO:243), gi478247382 (SEQ ID NO:244), gi482527365 (SEQ ID NO:245), gi490721105 (SEQ ID NO:246), gi493986068 (SEQ ID NO:247), gi499258510 (SEQ ID NO:248), gi499258936 (SEQ ID NO:249), gi501433637 (SEQ ID NO:250), gi501433833 (SEQ ID NO:251), gi503582056 (SEQ ID NO:252), gi503582195 (SEQ ID NO:253), gi505237613 (SEQ ID NO:254), gi505238317 (SEQ ID NO:255), gi516982302 (SEQ ID NO:256), gi516982304 (SEQ ID NO:257), gi516982708 (SEQ ID NO:258), gi516983121 (SEQ ID NO:259), gi516987696 (SEQ ID NO:260), gi517794814 (SEQ ID NO:261), gi517795927 (SEQ ID NO:262), gi518890617 (SEQ ID NO:263), gi519096608 (SEQ ID NO:264), gi519097773 (SEQ ID NO:265), gi519097774 (SEQ ID NO:266), gi519097795 (SEQ ID NO:267), gi519112703 (SEQ ID NO:268), gi543983601 (SEQ ID NO:269), gi545423791 (SEQ ID NO:270), gi545424583 (SEQ ID NO:271), gi545425072 (SEQ ID NO:272), gi545426533 (SEQ ID NO:273), gi545427051 (SEQ ID NO:274), gi545439371 (SEQ ID NO:275), gi545441087 (SEQ ID NO:276), gi545442195 (SEQ ID NO:277), gi547189063 (SEQ ID NO:278), gi548168374 (SEQ ID NO:279), gi550276358 (SEQ ID NO:280), gi557068 (SEQ ID NO:281), gi563394639 (SEQ ID NO:282), gi639227118 (SEQ ID NO:283), gi648615935 (SEQ ID NO:284), gi655448683 (SEQ ID NO:285), gi655944724 (SEQ ID NO:286), gi658545847 (SEQ ID NO:287), gi658546598 (SEQ ID NO:288), gi661258709 (SEQ ID NO:289), gi661305608 (SEQ ID NO:290), gi661309879 (SEQ ID NO:291), gi661310225 (SEQ ID NO:292), gi695722113 (SEQ ID NO:293), gi700231728 (SEQ ID NO:294), gi700260106 (SEQ ID NO:295), gi702977158 (SEQ ID NO:296), gi7245522 (SEQ ID NO:297), gi736404329 (SEQ ID NO:298), gi736404343 (SEQ ID NO:299), gi736408694 (SEQ ID NO:300), gi736412763 (SEQ ID NO:301), gi742261431 (SEQ ID NO:302), gi746372602 (SEQ ID NO:303), gi746373224 (SEQ ID NO:304), gi746373303 (SEQ ID NO:305), gi746375041 (SEQ ID NO:306), gi746375940 (SEQ ID NO:307), gi746376969 (SEQ ID NO:308), gi746377383 (SEQ ID NO:309), gi746377430 (SEQ ID NO:310), gi746382143 (SEQ ID NO:311), gi746383321 (SEQ ID NO:312), gi746385471 (SEQ ID NO:313), gi746385472 (SEQ ID NO:314), gi746385768 (SEQ ID NO:315), gi746388156 (SEQ ID NO:316), gi746388231 (SEQ ID NO:317), gi746390048 (SEQ ID NO:318), gi746391175 (SEQ ID NO:319), gi746391927 (SEQ ID NO:320), gi746392047 (SEQ ID NO:321), gi746395887 (SEQ ID NO:322), gi746395986 (SEQ ID NO:323), gi746398817 (SEQ ID NO:324), gi746399521 (SEQ ID NO:325), gi746402919 (SEQ ID NO:326), gi746403746 (SEQ ID NO:327), gi746404117 (SEQ ID NO:328), gi746404567 (SEQ ID NO:329), gi746404716 (SEQ ID NO:330), gi748669984 (SEQ ID NO:331), gi749914020 (SEQ ID NO:332), gi75345025 (SEQ ID NO:333), gi75348574 (SEQ ID NO:334), gi754467437 (SEQ ID NO:335), gi754467963 (SEQ ID NO:336), gi755775 (SEQ ID NO:337), gi762205553 (SEQ ID NO:338), gi762205668 (SEQ ID NO:339), gi762955586 (SEQ ID NO:340), gi762959862 (SEQ ID NO:341), gi762960358 (SEQ ID NO:342), gi763004002 (SEQ ID NO:343), gi763013925 (SEQ ID NO:344), gi763016605 (SEQ ID NO:345), gi763369462 (SEQ ID NO:346), gi763423212 (SEQ ID NO:347), gi763423514 (SEQ ID NO:348) or gi807048103 (SEQ ID NO:349).

In other embodiments, the immunogenic component may include peptidases or peptidase domains as included in or derived from the following Accession numbers or sequences having at least 90, 91, 92, 93, 94, 95, 96, 97, 98 or 99% or greater, identity thereto: gi505238317, gi516983121, gi763423212, gi167729944, gi516982302, gi661258709, gi518890617, gi661310225, gi655448683, gi517795927, gi746385471, gi563394639, gi746392047, gi746404716, gi746390048, gi746404117, gi742261431, gi746377430, gi763016605, gi695722113, gi700231728, gi545425072, gi545441087, gi700260106, gi545424583, gi545426533, gi746383321, gi392611916, gi545439371, gi482527365, gi762959862, gi746373303, gi501433833, gi763013925, gi75345025, gi2182812, gi75348574, gi762205668, gi2738803, gi503582056, gi736404343, gi736408694, gi519097774, gi648615935, gi45593253, gi1890077, gi655944724, gi519096608, gi519097795, gi754467437, gi2827775, gi516987696, gi763004002, gi4103639, gi746372602, gi762205553, gi188595513, gi557068, gi762955586, gi333803263, gi503582195, gi543983601, gi763423514, gi499258510, gi1066835, gi1813996, gi499258936, gi736412763, gi7245522, gi1246379, gi516982708, gi746373224, gi519112703, gi478247382, gi547189063, gi505237613, gi516982304, gi167730167, gi501433637, gi763369462, gi550276358, gi746395986, gi746403746, gi748669984, gi746376969, gi363646003, gi517794814, gi746382143, gi746375940, gi746391175, gi746388231, gi746385768, gi807048103, gi746398817, gi746375041, gi548168374, gi754467963, gi746377383, gi746395887 or gi746385472.

In still other embodiments, the immunogenic component may include peptidases or peptidase domains as included in or derived from the following Accession numbers, or have at least 90, 91, 92, 93, 94, 95, 96, 97, 98 or 99% or greater, identity thereto: gi499258510, gi746373224, gi501433637, gi1246379, gi478247382, gi545423791, gi545427051, gi545442195, gi7245522, gi762955586, gi1813996, gi333803263, gi1066835, gi499258936, gi763004002, gi2827775, gi490721105, gi543983601, gi188595513, gi746372602, gi557068, gi762205553, gi4103639, gi503582195, gi755775, gi746395986, gi517794814, gi746403746, gi746398817, gi746391175, gi746388231, gi746385768, gi746382143, gi746375940, gi746376969, gi807048103, gi746391927, gi746399521, gi746388156, gi746404567, gi702977158, gi746377383, gi746395887, gi746375041, gi746385472, gi746402919, gi298274962, gi749914020, gi736404329, gi658546598, gi545424583, gi545426533, gi545439371, gi763013925, gi762959862, gi482527365, gi392611916, gi742261431, gi695722113, gi762205668, gi2182812, gi75348574, gi503582056, gi2738803, gi45593253, gi746373303, gi75345025, gi658545847, gi545425072, gi545441087, gi563394639, gi763016605, gi700231728, gi746377430, gi746404716, gi746404117, gi746390048, gi517795927, gi746392047, gi746385471, gi501433833, gi700260106, gi1890077, gi493986068, gi746383321, gi519097774, gi736408694, gi736404343, gi639227118, gi519097773, gi547189063, gi661309879, gi763423514, gi376316122, gi167730167, gi736412763, gi519112703, gi763369462, gi762960358, gi661305608.

In some embodiments, the immunogenic component may include the sequences as set forth the any one or more of the following polypeptide sequences, or have at least 90, 91, 92, 93, 94, 95, 96, 97, 98 or 99% or greater, identity thereto: SEQ ID NOs.: 110, 111, 112, 113, 120, 123, 124, 125, 130, 131, 132, 133, 135, 136, 137, 138, 143, 144, 145, 146, 147, 148, 221, 222, 223 or 224. In particular embodiments, the immunogenic component may comprise, consist of or consist essentially of a polypeptide having the sequence as set forth in any one or more of the following: SEQ ID NOs.: 110, 111, 112, 113, 120, 123, 124, 125, 130, 131, 132, 133, 135, 136, 137, 138, 143, 144, 145, 146, 147, 148, 221, 222, 223 or 224.

In some embodiments, components of S. mutans, S. salivarius, S. mutans, S. sanguinis and S. sobrinus, P. gulae, P. salivosa, P. gingivalis and P. denticanis, are used to elicit production of adherence-blocking antibodies for the production of therapeutic IgY antibodies. The components may be, for example, any of the following: gingipain-like enzymes, gingipains derived from P. gingivalis deposited as ATCC 33277, P. endodontalis F5 or F2, P. circumdentaria, P. gingivalis 381, and the like. Gingipain enzymes may include at least the following: RgpA, HagA, Kgp and homologs, paralogs, and orthologs thereof

The present invention is based, in part, on the finding that IgY antibodies raised against certain bacteria and bacterial antigens may be incorporated into dosage forms, which are effective in maintaining the oral health of animals, in particular companion animals.

In certain embodiments, the IgY are produced according to standard techniques. For example, one of the foregoing antigens associated with oral health compromising (OHC) microorganisms, is administered to hens and IgY is recovered from said hen's yolk. Hens may be exposed to the OHC antigen through an injection. Said injection triggers a humoral immune response that manifests itself initially by the production of specific IgY in the blood serum of the immunized hen, followed by its export in the yolk of laid eggs. IgY antibodies against oral health compromising (OHC) microorganisms may be recovered or otherwise used in about 6-7 days or about 6 to about 10 days after administration of the antigen.

In an embodiment, the first step of producing an antimicrobial IgY antibody effective against OHC microorganism is to select a protein, peptide, polysaccharides, or entire OHC microorganism. In the case of small antigens (MW below about 12,000 da), conjugation to a carrier protein (e.g., bovine gamma globulin) may be required. The antigen dose is also critical because too much or too little antigen can lead to suppression, sensitization, tolerance or other undesirable immunomodulatory effects. The recommended amount of a soluble protein to be administered in a given vaccine dose is usually in the range of 0.01 mg to 1 mg.

Accordingly, in an advantageous embodiment, antimicrobial IgY antibodies according to the instant disclosure may be produced by administering to a hen one of the OHC microorganism-associated antigens recited above. For example, hens may be injected with an effective amount of a peptide having a similar immunogenic potential as compared with a peptide having the sequence as set forth in any one or more of SEQ ID NOs: 110-113, 120, 123-125, 130-133, 135-138, 143-148, 221-224 or 227-349.

In certain embodiments, veterinary compositions comprising the antimicrobial IgY antibodies of the invention are advantageously in the form of soft chewable formulations that are palatable for animals, including cats and dogs. In another embodiment, the oral veterinary compositions of the invention are in the form of a chewable tablet.

In other embodiments, the veterinary compositions may be oral ointments, gels, mouthwashes or liquids. Liquid formulations comprising IgY antibodies may be spray-deposited, or otherwise applied as a coating, to solid dosage forms, including chews and tablets, or may be applied to solid articles, such as chews or toys.

According to an aspect of the invention, an IgY antibody is used in the manufacture of a medicament for the prevention or treatment of a disease caused by oral bacteria in an animal.

According to a second aspect of the invention, a cosmetic method of preventing or decreasing oral staining, dental caries, or bad breath in an animal comprises the step of exposing the oral cavity to an IgY antibody raised against an antigen from or derived from a microorganism that causes oral health problems.

According to a third aspect of the invention, an animal chew comprises an IgY antibody raised against an antigen from or derived from a microorganism that causes oral health problems in animals, including humans, or a medicament comprising the IgY antibody.

According to a fourth aspect of the invention, an animal foodstuff comprises an IgY antibody raised against an antigen from or derived from a microorganism that causes oral health problems in animals, including humans.

### DETAILED DESCRIPTION OF THE INVENTION

The IgY antibodies are useful in preventing and treating diseases caused by oral bacteria in an “animal”. As used herein, the term “animal” is to be given its recognized meaning in the art, i.e. any non-human member of the animal kingdom. Preferably, the animal is a non-human mammal.

In a preferred embodiment, the animal is a “companion animal”. As used herein, the term “companion animal” refers to an animal that is kept as a “pet” by a person for companionship and enjoyment. These will usually be mammals such as cats, dogs, rabbits, ferrets and rodents. The most preferred companion animals are cats and dogs.

In an alternative embodiment, the animal is “livestock”, i.e. an animal that is reared agriculturally to provide a useful product. A livestock animal is usually a mammal, including but not limited to pigs, cows, goats, donkeys, sheep and llamas.

In another alternative embodiment, the animal is a “performance animal” such as a racehorse or racing greyhound. The skilled person will recognize the need for these animals to be maintained in optimum health, including oral health.

The IgY antibodies have been found to be particularly effective in maintaining the health and favorable aesthetics of an animal's oral cavity. The IgY antibodies have several medical (i.e. veterinary) applications, as follows: prevent the adherence of certain bacteria (known to promote dental caries) to an animal's teeth; prevent and remove plaque and calculus formation in the oral cavity, particularly on the teeth; prevent and treat gum diseases including gingivitis and periodontitis; and prevent and treat halitosis.

Surprisingly, polyclonal IgY antibodies, raised against antigens, including proteins, including native proteins and recombinant proteins, including recombinant proteins lacking any posttranslational modification, made by or derived from microorganisms that cause dental health problems, are more effective than corresponding monoclonal antibodies raised against the same antigens. In an embodiment, the polyclonal IgY antibodies are more effective at inhibiting the adherence of dental health problem-causing microorganisms than are monoclonal antibodies raised against the same antigens as were the polyclonal antibodies.

An IgY antibody may be incorporated into a medicament, composition or formulation. When an IgY antibody according to the invention is manufactured as a medicament, the skilled person will realize that the IgY antibody may be the only active ingredient in the final medicament. However, the final medicament may contain other pharmaceutically acceptable ingredients, both inert (i.e. an “excipient”) and pharmaceutically active. Any combination of an IgY antibody and one or more of the pharmaceutically acceptable ingredients disclosed below is within the scope of the invention. When the IgY antibody is prepared as a cosmetic, excipients may be added, which will be apparent to one skilled in the art.

The IgY antibody may be administered to an animal in combination with at least one anti-microbial, preferably anti-bacterial, agent. Suitable agents include one or more compound selected from Ferric Quinate (Akeso Biomedical; “Fe-QA,” also referred to herein as “FeQ”), and ferric complexes with L-tyrosine (Fe-Tyr), L-DOPA (Fe-DOPA), L-phenyl alanine (Fe-Phe) and hydrates, salts, or derivatives thereof. The agents may also include the antibiotics tetracycline, doxycycline and ampicillin and the anti-bacterial agents triclosan (2,4,4′-trichloro 2′-hydroxy diphenyl ether) and Chlorhexidine digluconate (1,1′-hexamethylene-bis[(5-p-chlorophenyl)-biguanide]). The IgY antibody is useful in treating and preventing gum disease and associated inflammation of the gums. The IgY antibody may be administered to an animal in combination with at least one anti-inflammatory agent. Anti-inflammatory agents are well known in the art and any may be used. Preferably, the anti-inflammatory agent is a non-steroidal anti-inflammatory drug (NSAID), such as aspirin (acetylsalicylic acid) or ibuprofen. In an alternative embodiment, a steroidal anti-inflammatory agent, for example cortisone, may be used.

The IgY antibody may also be co-administered with delmopinol.

The IgY antibody can be added (including by spray or other coating techniques) to chewable dosage forms, toothpaste, dentifrice, gum, gel or mouthwash formulations. The concentration of the IgY antibody that is required will vary depending on the animal to be treated and the nature of the formation, as will be apparent to the skilled person. An example of a suitable concentration of the preferred antibody is about 25 mg antibody per dose or application.

In a particular embodiment, the compound is present in an animal chew. As used herein, the term. “chew” is given its normal meaning in the art and refers to any toy, accessory or foodstuff that is intended for chewing or gnawing by an animal. The skilled person will recognize that suitable “chew” size and composition will vary depending on the animal. Chews may be made from animal products such as hide (animal skin), tendon or bone, man-made products such as plastics (e.g. nylon) and man-made rubber, and plant products such as natural rubber. Other suitable materials for making a chew will be apparent to the skilled person. A combination of materials may be used. Preferably, the chew provides sufficient resistance to the chewing action of an animal that pressure is put on the animal's teeth. It also acts to remove debris, plaque and/or calculus by friction.

The chew may be in any shape, for example, a chew made for a cat may be in the shape of a mouse and a chew for a dog may be in the shape of a bone. In a particular embodiment, the chew is shaped so that the teeth, gums and tongue are rubbed or massaged by the chew as the animal chews it, for example the chew may have a surface containing bumps, nodules or ridges. This will aid plaque removal and encourage uniform coating of the teeth by the IgY active ingredient.

In a particular embodiment, an IgY antibody is infused into the chew, i.e. the chew material contains the IgY antibody and releases it over a sustained period. An example of a method of infusing a chew is to soak hide in a stabilizing solution or suspension containing the IgY antibody, and then allow the hide to dry. In an alternative embodiment, the chew is simply coated with a stabilized IgY antibody. The chew may be flavored with a flavoring that is appealing to the animal it is designed for. As an example, a dog chew may be flavored with meat flavors, such as chicken or beef

The IgY antibody may alternatively be incorporated into a foodstuff that does not resist the chewing or gnawing action of an animal, i.e. foodstuffs that are readily broken down by the chewing or gnawing action. Preferred examples of such foodstuffs include animal feeds (both wet and dry), more particularly animal biscuits. The IgY antibody can be incorporated into, or coated onto the surface of, such foodstuffs. The IgY antibody could also be added to the animal's usual feed.

The chew or foodstuff comprising an IgY antibody can additionally comprise any of the other medicament ingredients disclosed herein, in any combination. For example, an animal foodstuff or chew can contain an IgY antibody and one or more active or inert pharmaceutical ingredients, such as an antimicrobial agent or an anti-inflammatory agent. The chew or foodstuff could also contain delmopinol.

In one embodiment, an IgY antibody is used to remove or prevent microorganisms that cause dental carries from adhering to an animal's teeth. In such an embodiment, the IgY antibody, or composition comprising the IgY antibody will have the effect of preventing plaque formation in the oral cavity, including the teeth and gums, of an animal. Prevention of plaque formation will prevent the formation of tartar (calculus), which forms when plaque calcifies and hardens. The terms “plaque” and “calculus” are given their normal meaning in the art. For the avoidance of doubt, plaque is a bacteria-based film that can occur on oral surfaces. Calculus (also known as tartar) is a hardened deposit that can form when plaque is not removed.

The two major forms of gum disease, gingivitis and periodontitis, result from bacterial plaque formation in the oral cavity, in particular on the teeth and gums. Although long considered to be a localized infection only, gum disease is now being investigated as a potential risk factor for the development of other, potentially more serious, diseases including cardiovascular disease and pulmonary disorders.

Therefore, IgY antibodies can be used to treat (by removing plaque) and prevent (by preventing plaque formation) gum diseases such as gingivitis and periodontitis. In a preferred embodiment, the gum disease has symptoms including infected sub-gingival pockets. More preferably, the infected sub-gingival pockets are at least 4 mm deep, measured from the tip of the gum line to the apex of the pocket.

A IgY antibody can be used to treat and prevent halitosis. As used herein, the term “halitosis” refers to the commonly recognized meaning of the term, i.e. “bad breath”. This may be defined as breath that has an odor that is unpleasant or offensive to the animal exhaling the breath, or more likely to people near the animal such as the owner. In a preferred embodiment, the breath contains Volatile Sulfur Compounds (VSC's), including but not limited to hydrogen sulfide, methyl mercaptan or dimethylsulfide, or compounds such as putrescine, cadaverine, butyric and propionic acids. These compounds result from proteolytic degradation of various sulfur-containing substrates in food debris, saliva, blood and epithelial cells, by predominantly anaerobic Gram-negative microorganisms in the oral cavity.

Thus, in an embodiment, an IgY antibody can reduce the level of these compounds emitted from an animal's mouth, thereby improving the animal's breath.

A composition comprising an IgY antibody can be used purely cosmetically, to reduce and prevent staining in the oral cavity. In particular, an IgY antibody can whiten the teeth by reducing the number of stain-causing bacteria. Any combination of an IgY antibody and one or more excipients, as described herein, is within the scope of this embodiment.

For all embodiments described herein, the oral cavity is exposed to (i.e. brought into contact with) an IgY antibody. The IgY antibody is brought into contact with the oral cavity in a conventional way, in any suitable form or amount that achieves the desired effect in the oral cavity, i.e. the reduction or prevention of plaque formation, prevention or treatment of gum diseases including gingivitis and periodontitis including treatment of infected sub-gingival pockets; prevention or treatment of halitosis or teeth whitening.

In some embodiments, the IgY antibody is in the form of a chewable dosage form, an aqueous mouthwash, toothpaste, gel, gum, dentifrice or other similar preparation that will be apparent to the skilled person.

In another embodiment, the IgY antibody contacts the oral cavity via its presence on or in a chew or foodstuff, as detailed above.

In one embodiment, whichever form is suitable, the IgY or IgY-containing composition held in the mouth for at least about 5 seconds, greater than about 10 seconds, or more than about one minute, two minutes, or more than several minutes.

In a particular embodiment, mechanical agitation, preferably brushing or scraping the teeth, tongue or gums, is performed simultaneously with or shortly, preferably immediately, after contacting the oral cavity with an IgY antibody. However, mechanical agitation is not required for the IgY antibodies to be effective in maintaining the health and favorable aesthetics of an animal's oral cavity.

Simple contact of the oral cavity with an IgY antibody is sufficient for the benefits described herein to be obtained. In one embodiment, the IgY antibody is applied as an aqueous mouthwash at the start of any regular (e.g. daily) oral health routine, such as before brushing the teeth. In a particular embodiment, the mouthwash is applied as a spray or mist, for example an aerosol spray, or as droplets from a dropper bottle. In a more particular embodiment, the oral cavity is contacted with an IgY antibody shortly, preferably immediately, before contacting the oral cavity with a further agent that is helpful in maintaining good oral conditions, for example a further de-staining agent.

A method for treating (veterinary or cosmetically) any of the oral health conditions disclosed herein comprises contacting the oral cavity of an animal that displays symptoms of one or more of the conditions with an IgY antibody, in particular, a preparation, composition or formulation containing an IgY antibody such as a toothpaste, gum, gel, dentifrice, mouthwash formulation, chew or foodstuff Halitosis, plaque formation, calculus and gum disease can be prevented by contacting the oral cavity with an IgY antibody, preferably a preparation containing an IgY antibody such as a toothpaste, gum, gel, mouthwash formulation, chew or foodstuff, prior to the development of offensive odors, plaque, calculus or disease.

In one embodiment of the invention, the veterinary compositions are in the form of a soft chewable composition. In another embodiment of the invention, the oral veterinary compositions are in the form of a chewable tablet. Each of the compositions of the invention is palatable to the animal and provides for easy administration of the composition to the animal. These compositions provide surprisingly effective protection of the animals against microorganisms that compromise their oral-health, for an extended period of time, while also providing a fast onset of action.

### DEFINITIONS

Terms used herein will have their customary meaning in the art unless specified otherwise.

The term “animal” is used herein to include all mammals, birds and fish and also include all vertebrate animals. Animals include, but are not limited to, cats, dogs, cattle, chickens, turkeys, deer, goats, horses, llamas, pigs, sheep, yaks, rodents and birds. It also includes an individual animal in all stages of development, including embryonic and fetal stages. In some embodiments, the animal will be a non-human animal.

The term “oral health compromising component (OHCC)” means a component of a microorganism, including the entire microorganism itself, which plays a significant role in the establishment of oral health diseases or pathologies, including, but not solely, the establishment of dental plaque, dental caries and gingivitis.

An “immunogenic oral health compromising component (IOHCC)” is thus an OHCC that is capable of eliciting an immune response when administered to an animal. In an advantageous embodiment, immunogenic OHCCs are administered to avian animals, including hens, to elicit production of OHCC-specific IgY antibodies. These antibodies have antimicrobial activity, particularly in that they are able to block microbial adherence to oral structures including teeth. As used herein, “antimicrobial” is intended to encompass “microbiocidal,” “microbiostatic,” “anti-adhesive,” “bacteria-inhibiting,” “bacteria-blocking,” and the like, which have their ordinary meanings. Accordingly, the Antimicrobial IgY Antibodies of the instant disclosure are useful for the treatment and prevention of oral health diseases and pathologies, including, but not solely, the establishment of dental plaque, dental caries and gingivitis. IOHCC naturally include polypeptides, antigens, immunogens and epitopes.

The expression “effective amount” as used herein means a concentration of the active agent in the composition sufficient to elicit the desired biological response to the target microorganism(s) after administration of the composition to the animal, as measured by methods known in the art and/or described in the examples herein. In some embodiments, an “effective amount” of the active agent in the composition will provide an efficacy of at least 70% against the target microorganism compared to an untreated control. In other embodiments, “an effective amount” of the active agent will provide an efficacy of at least 80%, or at least 85% compared to untreated controls. More typically, “an effective amount” of the active agent will provide an efficacy of at least 90%, at least 93%, at least 95% or at least 97% against the target microorganism. In certain embodiments, including the prevention of dental disease caused by S. mutans, the term “effective amount” may provide efficacy as high as 100%.

As used herein, the terms “systemically-acting” or “systemically active” will be understood to mean that the active components are active when administered orally and may be distributed through the plasma and/or tissues of the animal treated and act on the parasite when a blood meal is taken or when the parasite comes in contact with the active agent.

As used herein, the terms “locally-acting” or “locally active” will be understood to mean that the active components are active at the site of administration, and do not become extensively (or to any extent) distributed through the plasma and/or tissues of the animals.

As used herein, the term “amylaceous ingredients” is meant those food-stuffs containing a preponderance of starch and/or starch-like material. Examples of amylaceous ingredients are cereal grains and meals or flours obtained upon grinding cereal grains such as corn, oats, wheat, milo, barley, rice, and the various milling by-products of these cereal grains such as wheat feed flour, wheat middlings, mixed feed, wheat shorts, wheat red dog, oat groats, hominy feed, and other such material. Also included as sources of amylaceous ingredients are the tuberous food stuffs such as potatoes, tapioca, and the like.

As used herein the term “palatable” means an oral veterinary composition that is readily accepted by dogs without any coaxing or with limited coaxing. Palatable compositions are compositions that are consumed by at least 75% of dogs without manual administration of the composition.

The soft chewable veterinary compositions of the invention comprising at least one IgY active agent have been found to exhibit a very fast onset of action against microorganisms that harm animals, particularly, the dental and oral health of animals. For example, in some embodiments of the invention, the soft chewable veterinary compositions of the invention provide an efficacy of at least about 15%, at least about 20% or at least about 30% against S. mutans only about 30 minutes after administration to the animal compared with untreated controls, as measured according to the methods described in the examples.

In other embodiments, the soft chewable compositions of the invention provide an efficacy of at least about 30%, at least about 40% or at least about 50% against Streptococcus spp. only about 4 hours after administration. In still other embodiments, the compositions of the invention provide an efficacy of at least about 50%, at least about 60% or at least about 70% against fleas about 8 hours after administration to the animal. In yet other embodiments, the compositions of the invention provide an efficacy of at least about 85%, at least about 90%, at least about 95% or at least about 98% about 12 hours after administration of the composition to the animal. This surprisingly fast onset of efficacy is very important for effectively treating animals with established severe dental infections.

Typically, the IgY active agents may be present in the composition at a concentration of about 0.1 to about 40% (w/w). In another embodiment, the concentration of the IgY (s) active agents is about 0.1 to about 30% (w/w). In some embodiments of the invention, the IgY active agents are present in the composition at a concentration from about 1 to about 25% (w/w), about 1 to about 20% (w/w), about 1 to about 10% (w/w), about 1 to about 5% (w/w), or about 1 to about 3% (w/w). In still other embodiments, the IgY(s) active agents are present in a concentration of about 0.1 to about 5% (w/w), about 0.5 to about 5% (w/w), about 0.5 to about 3% (w/w) or about 1 to about 3% (w/w) in the composition. In yet other embodiments, the IgY(s) active agents are present in a concentration of about 3 to about 6% (w/w), or about 5 to 10% (w/w). In other embodiments, the IgY active agent is present in a relatively higher concentration in the dosage form, including about 5% (w/w) to about 15% (w/w), about 10% (w/w) to about 20% (w/w), about 10% (w/w) to about 15% (w/w) or about 15% (w/w) to about 20% (w/w) in the composition.

Some dosage units may contain from about 0.5 mg to about 2000 mg of at least one IgY active agent or a combination of IgY active agents. In one embodiment, the IgY active is present in an amount of from about 1 mg to about 200 mg in the composition. More typically, the IgY active agent is present in an amount of about 1 mg to about 150 mg or about 10 mg to about 150 mg per chewable unit. In some embodiments, the amount of at least one IgY active agent in a dosage unit is about 5 mg to about 50 mg, bout 1 mg to about 30 mg, or about 5 mg to about 30 mg. In other embodiments, the amount of at least one IgY active agent in a dosage unit of the invention is about 1 mg to about 20 mg or about 1 mg to about 15 mg. In other embodiments, the dosage units will contain about 50 mg to about 150 mg, about 50 mg to about 100 mg, or about 75 mg to about 140 mg of at least one IgY active agent.

In other embodiments, the amount of at least one IgY active agent will be about 100 mg to about 2000 mg per dosage unit. More typically, the amount of at least one IgY active agent in a dosage unit will be about 100 mg to about 1500 mg, about 100 mg to about 1000 mg or about 500 mg to about 1200 mg per dosage unit.

### Additional Active Agents

In another aspect of the invention, oral IgY veterinary compositions, including soft chewable compositions and chewable tablet compositions, that comprise one or more locally-acting Antimicrobial IgY Antibody active agent(s) are provided. Various classes of nutraceuticals, parasiticides and the like, and may be included in the oral veterinary compositions of the invention.

In one embodiment, the invention provides a soft chewable veterinary composition comprising at least one Antimicrobial IgY Antibody active agent in combination with at least one systemically-acting active agent that is active against endoparasites, and a pharmaceutically acceptable carrier or diluent. In another embodiment, the invention provides a soft chewable veterinary composition comprising at least one Antimicrobial IgY Antibody active agent in combination with at least one systemically-acting active agent that is active against ectoparasites, together with a pharmaceutically acceptable carrier or diluent.

In some embodiments, the additional active agents combined with an Antimicrobial IgY Antibody active agent may include, but are not limited to, acaricides, anthelmintics, insecticides and other parasiticides of various classes presented herein.

In another embodiment, the soft chewable compositions may also include veterinary therapeutic agents. Veterinary pharmaceutical agents that may be included in the compositions of the invention are well-known in the art (see e.g. Plumb′ Veterinary Drug Handbook, 5th Edition, ed. Donald C. Plumb, Blackwell Publishing, (2005) or The Merck Veterinary Manual, 9th Edition, (January 2005)) and include but are not limited to co-enzyme Q10, acarbose, acepromazine maleate, acetaminophen, acetazolamide, acetazolamide sodium, acetic acid, acetohydroxamic acid, acetylcysteine, acitretin, acyclovir, albendazole, albuterol sulfate, alfentanil, allopurinol, alprazolam, altrenogest, amantadine, amikacin sulfate, aminocaproic acid, aminopentamide hydrogen sulfate, aminophylline/theophylline, amiodarone, amitriptyline, amlodipine besylate, ammonium chloride, ammonium molybdenate, amoxicillin, clavulanate potassium, amphotericin B desoxycholate, amphotericin B lipid-based, ampicillin, amprolium, antacids (oral), antivenin, apomorphione, apramycin sulfate, ascorbic acid, asparaginase, aspiring, atenolol, atipamezole, atracurium besylate, atropine sulfate, aurnofin, aurothioglucose, azaperone, azathioprine, azithromycin, baclofen, barbituates, benazepril, betamethasone, bethanechol chloride, bisacodyl, bismuth subsalicylate, bleomycin sulfate, boldenone undecylenate, bromides, bromocriptine mesylate, budenoside, buprenorphine, buspirone, busulfan, butorphanol tartrate, cabergoline, calcitonin salmon, calcitrol, calcium salts, captopril, carbenicillin indanyl sodium, carbimazole, carboplatin, carnitine, carprofen, carvedilol, cefadroxil, cefazolin sodium, cefixime, clorsulon, cefoperazone sodium, cefotaxime sodium, cefotetan disodium, cefoxitin sodium, cefpodoxime proxetil, ceftazidime, ceftiofur sodium, ceftiofur, ceftiaxone sodium, cephalexin, cephalosporins, cephapirin, charcoal (activated), chlorambucil, chloramphenicol, chlordiazepoxide, chlordiazepoxide+/−clidinium bromide, chlorothiazide, chlorpheniramine maleate, chlorpromazine, chlorpropamide, chlortetracycline, chorionic gonadotropin (HCG), chromium, cimetidine, ciprofloxacin, cisapride, cisplatin, citrate salts, clarithromycin, clemastine fumarate, clenbuterol, clindamycin, clofazimine, clomipramine, claonazepam, clonidine, cloprostenol sodium, clorazepate dipotassium, clorsulon, cloxacillin, codeine phosphate, colchicine, corticotropin (ACTH), cosyntropin, cyclophosphamide, cyclosporine, cyproheptadine, cytarabine, dacarbazine, dactinomycin/actinomycin D, dalteparin sodium, danazol, dantrolene sodium, dapsone, decoquinate, deferoxamine mesylate, deracoxib, deslorelin acetate, desmopressin acetate, desoxycorticosterone pivalate, detomidine, dexamethasone, dexpanthenol, dexraazoxane, dextran, diazepam, diazoxide (oral), dichlorphenamide, diclofenac sodium, dicloxacillin, diethylcarbamazine citrate, diethylstilbestrol (DES), difloxacin, digoxin, dihydrotachysterol (DHT), diltiazem, dimenhydrinate, dimercaprol/BAL, dimethyl sulfoxide, dinoprost tromethamine, diphenylhydramine, disopyramide phosphate, dobutamine, docusate/DSS, dolasetron mesylate, domperidone, dopamine, doramectin, doxapram, doxepin, doxorubicin, doxycycline, edetate calcium disodium.calcium EDTA, edrophonium chloride, enalapril/enalaprilat, enoxaparin sodium, enrofloxacin, ephedrine sulfate, epinephrine, epoetin/erythropoietin, eprinomectin, epsiprantel, erythromycin, esmolol, estradiol cypionate, ethacrynic acid/ethacrynate sodium, ethanol (alcohol), etidronate sodium, etodolac, etomidate, euthanasia agents w/pentobarbital, famotidine, fatty acids (essential/omega), felbamate, fentanyl, ferrous sulfate, filgrastim, finasteride, fipronil, florfenicol, fluconazole, flucytosine, fludrocortisone acetate, flumazenil, flumethasone, flunixin meglumine, fluorouracil (5-FU), fluoxetine, fluticasone propionate, fluvoxamine maleate, fomepizole (4-MP), furazolidone, furosemide, gabapentin, gemcitabine, gentamicin sulfate, glimepiride, glipizide, glucagon, glucocorticoid agents, glucosamine/chondroitin sulfate, glutamine, glyburide, glycerine (oral), glycopyrrolate, gonadorelin, grisseofulvin, guaifenesin, halothane, hemoglobin glutamer-200 (OXYGLOBIN®®), heparin, hetastarch, hyaluronate sodium, hydrazaline, hydrochlorothiazide, hydrocodone bitartrate, hydrocortisone, hydromorphone, hydroxyurea, hydroxyzine, ifosfamide, imidacloprid, imidocarb dipropinate, impenem-cilastatin sodium, imipramine, inamrinone lactate, insulin, interferon alfa-2a (human recombinant), iodide (sodium/potassium), ipecac (syrup), ipodate sodium, iron dextran, isoflurane, isoproterenol, isotretinoin, isoxsuprine, itraconazole, ivermectin, kaolin/pectin, ketamine, ketoconazole, ketoprofen, ketorolac tromethamine, lactulose, leuprolide, levamisole, levetiracetam, levothyroxine sodium, lidocaine, lincomycin, liothyronine sodium, lisinopril, lomustine (CCNU), lufenuron, lysine, magnesium, mannitol, marbofloxacin, mechlorethamine, meclizine, meclofenamic acid, medetomidine, medium chain triglycerides, medroxyprogesterone acetate, megestrol acetate, melarsomine, melatonin, meloxican, melphalan, meperidine, mercaptopurine, meropenem, metformin, methadone, methazolamide, methenamine mandelate/hippurate, methimazole, methionine, methocarbamol, methohexital sodium, methotrexate, methoxyflurane, methylene blue, methylphenidate, methylprednisolone, metoclopramide, metoprolol, metronidaxole, mexiletine, mibolerlone, midazolam milbemycin oxime, mineral oil, minocycline, misoprostol, mitotane, mitoxantrone, morphine sulfate, moxidectin, naloxone, mandrolone decanoate, naproxen, narcotic (opiate) agonist analgesics, neomycin sulfate, neostigmine, niacinamide, nitazoxanide, nitenpyram, nitrofurantoin, nitroglycerin, nitroprusside sodium, nizatidine, novobiocin sodium, nystatin, octreotide acetate, olsalazine sodium, omeprozole, ondansetron, opiate antidiarrheals, orbifloxacin, oxacillin sodium, oxazepam, oxibutynin chloride, oxymorphone, oxytretracycline, oxytocin, pamidronate disodium, pancreplipase, pancuronium bromide, paromomycin sulfate, parozetine, pencillamine, general information penicillins, penicillin G, penicillin V potassium, pentazocine, pentobarbital sodium, pentosan polysulfate sodium, pentoxifylline, pergolide mesylate, phenobarbital, phenoxybenzamine, pheylbutazone, phenylephrine, phenypropanolamine, phenytoin sodium, pheromones, parenteral phosphate, phytonadione/vitamin K-1, pimobendan, piperazine, pirlimycin, piroxicam, polysulfated glycosaminoglycan, ponazuril, potassium chloride, pralidoxime chloride, prazosin, prednisolone/prednisone, primidone, procainamide, procarbazine, prochlorperazine, propantheline bromide, propionibacterium acnes injection, propofol, propranolol, protamine sulfate, pseudoephedrine, psyllium hydrophilic mucilloid, pyridostigmine bromide, pyrilamine maleate, pyrimethamine, quinacrine, quinidine, ranitidine, rifampin, s-adenosyl-methionine (SAMe), saline/hyperosmotic laxative, selamectin, selegiline/1-deprenyl, sertraline, sevelamer, sevoflurane, silymarin/milk thistle, sodium bicarbonate, sodium polystyrene sulfonate, sodium stibogluconate, sodium sulfate, sodum thiosulfate, somatotropin, sotalol, spectinomycin, spironolactone, stanozolol, streptokinase, streptozocin, succimer, succinylcholine chloride, sucralfate, sufentanil citrate, sulfachlorpyridazine sodium, sulfadiazine/trimethroprim, sulfamethoxazole/trimethoprim, sulfadimentoxine, sulfadimethoxine/ormetoprim, sulfasalazine, taurine, tepoxaline, terbinafline, terbutaline sulfate, testosterone, tetracycline, thiacetarsamide sodium, thiamine, thioguanine, thiopental sodium, thiotepa, thyrotropin, tiamulin, ticarcilin disodium, tiletamine/zolazepam, tilmocsin, tiopronin, tobramycin sulfate, tocainide, tolazoline, telfenamic acid, topiramate, tramadol, trimcinolone acetonide, trientine, trilostane, trimepraxine tartrate w/prednisolone, tripelennamine, tylosin, urdosiol, valproic acid, vanadium, vancomycin, vasopressin, vecuronium bromide, verapamil, vinblastine sulfate, vincristine sulfate, vitamin E/selenium, warfarin sodium, xylazine, yohimbine, zafirlukast, zidovudine (AZT), zinc acetate/zinc sulfate, zonisamide and mixtures thereof

In one embodiment of the invention, arylpyrazole compounds such as phenylpyrazoles may be included in the oral veterinary compositions of the invention. The arylpyrazoles are known in the art and are suitable for combination with the isoxazoline compounds in the soft chewable compositions of the invention. Examples of such arylpyrazole compounds include but are not limited to those described in U.S. Pat. Nos. 6,001,384; 6,010,710; 6,083,519; 6,096,329; 6,174,540; 6,685,954, 6,998,131 and 7,759,381 (all of which are incorporated herein by reference). A particularly preferred arylpyrazole active agent is fipronil. In one embodiment, the arylpyrazole may be included in the soft chewable compositions in combination with one or more Antimicrobial IgY Antibody active agents, one or more macrocyclic lactones, one or more spinosyn compounds, one or more spinosoid compounds, a benzimidazole, levamisole, pyrantel, morantel, praziquantel, closantel, clorsulon, one or more amino acetonitrile active agent, one or more insect growth regulators, one or more neonicotinoids or one or more aryloazol-2-yl cyanoethylamino active agents, or a combination of thereof

In another embodiment of the invention, one or more macrocyclic lactones or lactams, which act as an acaricide, an anthelmintic agent and/or an insecticide, can be included in the compositions of the invention. The macrocyclic lactone active agents are very potent and may be included alone in the compositions or in combination with one or more Antimicrobial IgY Antibody active agents, one or more spinosyn compounds, one or more spinosoid compounds, a benzimidazole, levamisole, pyrantel, morantel, praziquantel, closantel, clorsulon, one or more amino acetonitrile active agent, one or more insect growth regulators, one or more neonicotinoids or one or more aryloazol-2-yl cyanoethylamino active agents, or a combination of thereof. Furthermore, in one embodiment, the oral veterinary compositions of the invention may comprise a combination of two or more macrocyclic lactone active agents, alone or in combination with other systemically-acting active agents. For the avoidance of doubt, the term “macrocyclic lactone” as used herein includes both naturally occurring and synthetic or semi-synthetic avermectin and milbemycin compounds.

The macrocyclic lactones that may be used in the compositions of the invention include, but are not limited to, the naturally produced avermectins (e.g. including the components designated as A1a, A1b, A2a, A2b, B1a, B1b, B2a and B2b) and milbemycin compounds, semisynthetic avermectins and milbemycins, avermectin monosaccharide compounds and avermectin aglycone compounds. Examples of macrocyclic lactone compounds that may be used in the compositions include, but are not limited to, abamectin, dimadectin, doramectin, emamectin, eprinomectin, ivermectin, latidectin, lepimectin, selamectin, ML-1,694,554 and milbemycins including, but not limited to, milbemectin, milbemycin D, milbemycin A3, milbemycin A4, milbemycin oxime, moxidectin and nemadectin. Also included are the 5-oxo and 5-oxime derivatives of said avermectins and milbemycins.

The macrocyclic lactone compounds are known in the art and can easily be obtained commercially or through synthesis techniques known in the art. Reference is made to the widely available technical and commercial literature. For avermectins, ivermectin and abamectin, reference may be made, for example, to the work “Ivermectin and Abamectin”, 1989, by M. H. Fischer and H. Mrozik, William C. Campbell, published by Springer Verlag., or Albers-Schonberg et al. (1981), “Avermectins Structure Determination”, J. Am. Chem. Soc., 103, 4216-4221. For doramectin, “Veterinary Parasitology”, vol. 49, No. 1, July 1993, 5-15 may be consulted. For milbemycins, reference may be made, inter alia, to Davies H. G. et al., 1986, “Avermectins and Milbemycins”, Nat. Prod. Rep., 3, 87-121, Mrozik H. et al., 1983, Synthesis of Milbemycins from Avermectins, Tetrahedron Lett., 24, 5333-5336, U.S. Pat. No. 4,134,973 and EP 0 677 054, both incorporated herein by reference.

The structure of the avermectins and milbemycins are closely related, e.g., by sharing a complex 16-membered macrocyclic lactone ring. The natural product avermectins are disclosed in U.S. Pat. No. 4,310,519 and the 22,23-dihydro avermectin compounds are disclosed in U.S. Pat. No. 4,199,569. Mention is also made of U.S. Pat. Nos. 4,468,390, 5,824,653, EP 0 007 812 A1, U.K. Patent Specification 1 390 336, EP 0 002 916, and New Zealand Patent No. 237 086, inter alia. Naturally occurring milbemycins are described in U.S. Pat. No. 3,950,360 as well as in the various references cited in “The Merck Index” 12th ed., S. Budavari, Ed., Merck & Co., Inc. Whitehouse Station, New Jersey (1996). Latidectin is described in the “International Nonproprietary Names for Pharmaceutical Substances (INN)”, WHO Drug Information, vol. 17, no. 4, pp. 263-286, (2003). Semisynthetic derivatives of these classes of compounds are well known in the art and are described, for example, in U.S. Pat. Nos. 5,077,308, 4,859,657, 4,963,582, 4,855,317, 4,871,719, 4,874,749, 4,427,663, 4,310,519, 4,199,569, 5,055,596, 4,973,711, 4,978,677, 4,920,148 and EP 0 667 054, all incorporated herein by reference.

In one embodiment, the oral veterinary compositions of the invention, including soft chewable compositions and chewable tablet compositions, comprise an effective amount of at least one of abamectin, dimadectin, doramectin, emamectin, eprinomectin, ivermectin, latidectin, lepimectin, selamectin, milbemectin, milbemycin D, milbemycin A3, milbemycin A4, milbemycin oxime, moxidectin or nemadectin, or a combination thereof. In another embodiment, the invention provides a soft chewable veterinary composition comprising an effective amount of at least one of abamectin, emamectin, eprinomectin, ivermectin, doramectin or selamectin, or a combination thereof. In still another embodiment, the soft chewable veterinary compositions of the invention comprise an effective amount of at least one of ivermectin, milbemectin, milbemycin oxime or moxidectin, or a combination thereof

In another embodiment, oral veterinary compositions comprising at least one Antimicrobial IgY Antibody active agent in combination with abamectin, dimadectin, doramectin, emamectin, eprinomectin, ivermectin, latidectin, lepimectin, selamectin, milbemectin, milbemycin D, milbemycin A3, milbemycin A4, milbemycin oxime, moxidectin or nemadectin, or a combination thereof, are provided. In still another embodiment, oral veterinary compositions comprising at least one Antimicrobial IgY Antibody active agent in combination with abamectin, emamectin, eprinomectin, ivermectin, doramectin or selamectin, or a combination thereof, are provided.

In yet another embodiment, soft chewable veterinary compositions comprising at least one Antimicrobial IgY Antibody active agent in combination with an effective amount of ivermectin, milbemectin, milbemycin oxime or moxidectin, or a combination thereof, are provided.

In another embodiment, the invention provides a soft chewable veterinary composition comprising an effective amount of at least one Antimicrobial IgY Antibody active agent in combination with an effective amount of abamectin, dimadectin, doramectin, emamectin, eprinomectin, ivermectin, latidectin, lepimectin, selamectin, milbemectin, milbemycin D, milbemycin A3, milbemycin A4, milbemycin oxime, moxidectin or nemadectin, or a combination thereof. In another embodiment, the invention provides a soft chewable veterinary composition comprising an effective amount of at least one Antimicrobial IgY Antibody active agent in combination with an effective amount of abamectin, emamectin, eprinomectin, ivermectin, doramectin or selamectin, or a combination thereof

In still another embodiment, the invention provides a soft chewable veterinary composition comprising an effective amount of at least one Antimicrobial IgY Antibody active agent in combination with an effective amount of at least one of ivermectin, milbemectin, milbemycin D, milbemycin A3, milbemycin A4, milbemycin oxime, moxidectin or nemadectin, or a combination thereof

In some embodiments, the chewable veterinary compositions may comprise a combination of at least one Antimicrobial IgY Antibody active agent with two different macrocyclic lactone active agents.

In still another embodiment, the invention provides a soft chewale veterinary composition comprising an effective amount of an anti-S. mutans IgY in combination with an effective amount of abamectin, emamectin, eprinomectin, ivermectin or selamectin, or a combination thereof. In yet another embodiment, the invention provides a soft chewable veterinary composition comprising an effective amount of an anti-S. mutans IgY in combination with an effective amount of ivermectin, milbemycin oxime or moxidectin, or a combination thereof.

### Formulations

In one embodiment of the invention, the soft chewable veterinary compositions are in the form of a soft chewable formulation (“soft chew”) which is palatable and acceptable to the animal. In addition to the active ingredient(s), the soft chews of the invention may include one or more of the following components: a solvent or mixture of solvents, one or more fillers, one or more binders, one or more surfactants, one or more humectants, one or more lubricants, one or more disintegrants, one or more colorants, one or more antimicrobial agents, one or more antioxidants, one or more pH modifiers and one or more flavoring agents.

Preferably, the components of the oral veterinary compositions will be classified as food grade quality or higher (e.g. USP or NF grade). The term “food grade” is used to refer to material that is suitable for consumption by animals and will not contain chemical or other agents that are hazardous to the health of the animal. Thus, a food grade component, if of animal origin, will be prepared to substantially reduce or eliminate the presence of infectious agents or contaminants by processes known in the art such as pasteurization, filtration, pressurization or irradiation. More preferably, the components of the soft chewable veterinary compositions of the invention will not be of animal origin to avoid transmission of infective agents.

Solvents that may be used in the compositions of the invention include, but are not limited to, various grades of liquid polyethylene glycol (PEG) including PEG 200, PEG 300, PEG 400 and PEG 540; propylene carbonate; propylene glycol; triglycerides including, but not limited to caprylic/capric triglyceride, caprylic/capric/linoleic triglyceride (e.g. MIGLYOL® 810 and 812, caprylic/capric/succinic triglyceride, propylene glycol dicaprylate/dicaprate, and the like; water, sorbitol solution, glycerol caprylate/caprate and polyglycolized glycerides (GELUCIRE®), or a combination thereof

Solvents may be included in the compositions in concentrations of about 1 to about 50% (w/w). In other embodiments, the concentration of the solvents will be from about 1 to about 40% (w/w), about 1 to about 30% (w/w) or about 1 to about 20% (w/w). More typically, the solvents will be in the compositions at concentrations of about 5% to about 20% (w/w) or about 5% to about 15% (w/w).

Various fillers known in the art may be used in the soft chewable compositions of the invention. Fillers include, but are not limited to, corn starch, pre-gelatinized corn starch, soy protein fines, corn cob, and corn gluten meal, and the like. In some embodiments, a combination of two or more fillers may be used in the compositions.

The starch component may comprise starch from any source and may act as a binder in the soft chew. In one embodiment, the starch component used in the compositions is unmodified. In another embodiment, the starch component is derivatized and/or pregelatinized. In another embodiment, the starch component is highly derivatized. Some starches that can serve as a base starch for derivatization include regular corn, waxy corn, potato, tapioca, rice, etc. Suitable types of derivatizing agents for the starch include, but are not limited to, ethylene oxide, propylene oxide, acetic anhydride, and succinic anhydride, and other food approved esters or ethers, introducing such chemicals alone or in combination with one another.

In various embodiments, prior cross-linking of the starch in the starch component may or may not be necessary, based on the pH of the system and the temperature used to form the product.

The starch component may also include amylaceous ingredients. The amylaceous ingredients can be gelatinized or cooked before or during the forming step to achieve the desired matrix characteristics. If gelatinized starch is used, it may be possible to prepare the product of the subject invention or perform the process of the subject invention without heating or cooking. However, ungelatinized (ungelled) or uncooked starch may also be used.

Fillers are typically present in the compositions at a concentration of about 5% to about 80% (w/w), about 10% to about 70% (w/w), about 10% to about 60%, about 10% to about 50% (w/w), or about 10% to about 40% (w/w). More typically, the fillers may be present at concentrations of about 30% to about 70%, about 30% to about 60%, about 30% to about 50% or about 35% to about 55%.

Binders that may be used in the compositions of the invention include, but are not limited to, polyvinylpyrrolidone (e.g. Povidone), cross-linked polyvinylpyrrolidone (Crospovidone), polyethylene glycols of various grades including PEG 3350, PEG 4000, PEG 6000, PEG 8000 and even PEG 20,000, and the like; co-polymers of vinylpyrrolidone and vinyl acetate (e.g. Copovidone) such as the product sold by BASF by the tradename Kollidon® VA 64 and the like; starch such as potato starch, tapioca starch or corn starch; molasses, corn syrup, honey, maple syrup and sugars of various types; or a combination of two or more binders. In one embodiment, the composition comprises the binders Povidone K30 LP and PEG 3350 or PEG 4000, or a combination thereof. Binders are typically present in the compositions at a concentration of about 1% to about 30% (w/w). More typically, the compositions will include binders at a concentration of about 1% to about 20% (w/w), about 1 to about 15% (w/w), about 1% to about 10% (w/w), about 5% to about 15% (w/w) or about 5% to about 10% (w/w).

Humectants that may be used in the compositions include, but are not limited to, glycerol (also referred to herein as glycerin), propylene glycol, cetyl alcohol and glycerol monostearate, and the like. Polyethylene glycols of various grades may also be used as humectants.

In some embodiments, the humectant may comprise more than one oil including, but not limited to, fat or fats, both natural and synthetic. Oil employed as an ingredient in the soft chew may be a saturated or unsaturated liquid fatty acid, its glyceride derivatives or fatty acid derivatives of plant or animal origin or a mixture thereof. A source for typical animal fats or oils are fish oil, chicken fat, tallow, choice white grease, prime steam lard and mixtures thereof. However, other animal fats are also suitable for use in the soft chew. Suitable sources for vegetable fats or oils can be derived palm oil, palm hydrogenated oil, corn germ hydrogenated oil, castor hydrogenated oil, cotton-seed oil, soybean oil, olive oil, peanut oil, palm olein oil, Cacao fat, margarine, butter, shortening and palm stearin oil, and mixtures thereof. Additionally, a mixture of animal or vegetable oils or fats is suitable for use in the matrix.

Humectants may typically present in the compositions at a concentration of about 1% to about 25% (w/w). Typically, the concentration of the humectant in the composition of the invention will be 1% to about 20% (w/w), about 1% to about 15% (w/w) or about 5% to about 15% (w/w). More typically, the compositions of the invention will contain about 1% to about 10% (w/w) humectant.

Surfactants may be present in the composition at concentrations of about 0.1% to about 10% (w/w), about 1% to about 10% (w/w) or about 5% to about 10% (w/w). More typically, surfactants may be present at concentrations of about 0.1% to about 5% (w/w) or about 1 to about 5% (w/w). Examples of surfactants that may be used in the compositions include, but are not limited to, glyceryl monooleate, polyoxyethylene sorbitan fatty acid esters, sorbitan esters including sorbitan monooleate (Span® 20), polyvinyl alcohol, polysorbates including polysorbate 20 and polysorbate 80, d-α-tocopheryl polyethylene glycol 1000 succinate (TPGS), sodium lauryl sulfate, co-polymers of ethylene oxide and propylene oxide (e.g. poloxamers such as LUTROL® F87 and the like), polyethylene glycol castor oil derivatives including polyoxyl 35 castor oil (Cremophor® EL), polyoxyl 40 hydrogenated castor oil (Cremophor® RH 40), polyoxyl 60 hydrogenated castor oil (Cremophor® RH60); propylene glycol monolaurate (LAUROGLYCOL®); glyceride esters including glycerol caprylate/caprate (CAPMUL® MCM), polyglycolized glycerides (GELUCIRE®), PEG 300 caprylic/capric glycerides (Softigen® 767), PEG 400 caprylic/capric glycerides (Labrasol®), PEG 300 oleic glycerides (Labrafil® M-1944CS), PEG 300 linoleic glycerides (Labrafil® M-2125CS); polyethylene glycol stearates and polyethylene glycol hydroxy stearates including polyoxyl 8 stearate (PEG 400 monostearate), polyoxyl 40 stearate (PEG 1750 monostearate, and the like. Polyethylene glycol stearates (synonyms include macrogol stearates, polyoxylstearates, polyoxyethylene stearates, ethoxylated stearates; CAS No. 9004-99-3, 9005-08-7) are mixtures of mono- and distearate esters of mixed polyoxyethylene polymers. Polyethylene glycol hydroxystearate is a mixture of mono- and diesters of hydroxystearic acid with polyethylene glycols. One polyethylene glycol hydroxystearate that may be used in the compositions is polyethylene glycol 12-hydroxystearate. In another embodiment, the compositions may include the surfactant polyethylene glycol 15 12-hydroxystearate (Solutol® HS 15 from BASF), a mixture of mono- and diesters of 12-hydroxystearic acid with 15 moles of ethylene oxide. Again, these compounds, as well as their amounts are well known in the art. In another embodiment of the invention, the compositions may include polyoxyl 35 castor oil (Cremophor® EL) as a surfactant. In other embodiments, the chewable compositions may include polyoxyl 40 hydrogenated castor oil (Cremophor® RH 40) or polyoxyl 60 hydrogenated castor oil (Cremophor® RH60) as surfactants. The compositions of the invention may also include a combination of surfactants.

The type and nature of the surfactant has been found to be very important in keeping the active agent(s) in solution after ingestion and dissolution of the oral compositions. This is particularly important for obtaining the very high bioavailability observed from the inventive oral compositions. However, it has been found that inclusion of certain surfactants with the veterinary dosage forms adversely affect the palatability of the dosage form, resulting in low acceptance by the animals treated. In one embodiment, polyethylene glycol 15 hydroxystearate, polyoxyl 40 hydrogenated castor oil or polyoxyl 60 hydrogenated castor oil, are effective for solubilizing active agents with low water solubility including, but not limited to, isoxazoline active agents and the like, after ingestion by the animal while also maintaining the palatability of the oral dosage form. Thus, in one embodiment of the invention, the oral veterinary compositions comprise Polyethylene glycol 15 hydroxystearate, polyoxyl 40 hydrogenated castor oil or polyoxyl 60 hydrogenated castor oil. In another embodiment of the invention, the veterinary soft chewable compositions of the invention comprise Polyethylene glycol 15 hydroxystearate, polyoxyl 40 hydrogenated castor oil or polyoxyl 60 hydrogenated castor oil at a concentration of about 1 to about 5% (w/w).

In some embodiments, the compositions of the invention may contain one or more disintegrants. Examples of disintegrants that may be used in the compositions of the invention include, but are not limited to, cellulose, carboxymethyl cellulose calcium, carboxymethyl cellulose sodium, polacrilin potassium, starch, hydroxypropyl starch, corn starch, pregelatinized starch, modified starch, lactose monohydrate, croscarmellose sodium, hydroxypropyl cellulose, glycine, Crospovidone, magnesium aluminum silicate, sodium starch glycolate, guar gum, colloidal silicon dioxide, polyvinylpyrrolidone (Povidone), alginic acid, sodium alginate, calcium alginate, methylcellulose, chitosan, and the like, or a combination thereof

In certain embodiments, the oral veterinary compositions of the invention will include up to about 10% (w/w) of one or more disintegrants. In one embodiment, the compositions may include about 1% (w/w) to about 7% (w/w) of one or more disintegrants. In another embodiment, the compositions may include about 1% (w/w) to about 5% (w/w) or about 2% (w/w) to about 4% (w/w) of one or more disintegrants.

The inventive formulations may contain other inert ingredients such as antioxidants, preservatives, or pH stabilizers. These compounds are well known in the formulation art. Antioxidants may be added to the compositions of the invention to inhibit degradation of the active agents. Suitable antioxidants include, but are not limited to, alpha tocopherol, ascorbic acid, ascorbyl palmitate, fumaric acid, malic acid, sodium ascorbate, sodium metabisulfate, n-propyl gallate, BHA (butylated hydroxy anisole), BHT (butylated hydroxy toluene) monothioglycerol and the like. The antioxidants are generally added to the formulation in amounts of from about 0.01 to about 2.0% (w/w), based upon total weight of the formulation, with about 0.05 to about 1.0% or about 0.1% to about 0.2% (w/w) being especially preferred.

The compositions of the invention may also include one or more lubricants/processing aids. In some cases, the lubricant/processing aid may also behave as a solvent, and accordingly, there some of the components of the inventive compositions may have dual functions. Lubricants/processing aids include, but are not limited to polyethylene glycols of various molecular weight ranges including PEG 3350 (Dow Chemical) and PEG 4000, corn oil, mineral oil, hydrogenated vegetable oils (STEROTEX or LUBRITAB), peanut oil and/or castor oil. In certain embodiments, the lubricant/processing aid is a neutral oil comprising a medium chain triglyceride or propylene glycol fatty acid esters including caprylic/capric triglycerides. Non-limiting examples of neutral oils are known by the trademark MIGLYOL® including MIGLYOL® 810, MIGLYOL® 812, MIGLYOL® 818, MIGLYOL® 829 and MIGLYOL® 840. If present, the lubricant/processing aid may be in the composition at a concentration of about 1% to about 20% (w/w). Typically, the lubricant/processing aid will be present at a concentration of about 1% to about 15% (w/w) or about 1% to about 10% (w/w). Preferably, the lubricant/processing aid will be present in the composition at a concentration of about 1% to about 5% (w/w).

The compositions may also include anti-microbial agents or preservatives. Suitable preservatives include, but are not limited to, the parabens (methylparaben and/or propylparaben), benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. The concentration of the preservatives in the compositions of the invention are typically from about 0.01 to about 5.0% (w/w), about 0.01 to about 2% (w/w) or about 0.05 to about 1.0% (w/w). In one embodiment, the compositions of the invention will contain about 0.1% to about 0.5% (w/w) of the preservative.

In an embodiment, the oral veterinary compositions of the invention may contain one or more stabilizers to stabilize active ingredients that are susceptible. Suitable stabilizer components include, but are not limited to, magnesium stearate, citric acid, sodium citrate, and the like. However, stabilizer components are common in the art and any suitable one or mixture of more than one may be used. In an embodiment, a stabilizer component comprises about 0.0 percent to about 3.0 percent of the soft chew. In an alternate embodiment, a stabilizer component comprises about 0.5 percent to about 1.5 percent of the soft chew.

Compounds which stabilize the pH of the formulation are also contemplated in the compositions of the invention. Again, such compounds are well known to a practitioner in the art as well as how to use these compounds. Buffering systems include, for example, systems selected from acetic acid/acetate, malic acid/malate, citric acid/citrate, tartaric acid/tartrate, lactic acid/lactate, phosphoric acid/phosphate, glycine/glycimate, tris, glutamic acid/glutamates and sodium carbonate. In one embodiment, the compositions may include the pH modifier citric acid or a citric acid/citrate combination. The amount of the pH modifier required to achieve a desired pH depends on the nature of the active ingredient(s) and non-active excipients. However, in some embodiments the pH modifier may typically be present in an amount of about 0.1 to about 5% (w/w), about 0.1 to about 3% (w/w) or about 0.1 to about 2% (w/w). More typically, the pH modifier may be present in a concentration of about 0.1 to 1% (w/w) in the inventive compositions.

Many flavoring agents may be used in the compositions of the invention to improve the palatability of the oral veterinary formulations. Preferred flavoring agents are those that are not derived from animal sources. In various embodiments, flavoring components derived from fruit, meat (including, but not limited to pork, beef, chicken, fish, poultry, and the like), vegetable, cheese, bacon, cheese-bacon and/or artificial flavorings may be used. A flavoring component is typically chosen based upon consideration related to the organism that will be ingesting the soft chew. For example, a horse may prefer an apple flavoring component, while a dog may prefer a meat flavoring component. Although flavoring components derived from non-animal sources are preferred, in some embodiments, natural flavors containing beef or liver extracts, etc., may be used such as braised beef flavor artificial powdered beef flavor, roast beef flavor and corned beef flavor among others.

Non-animal flavoring agents include, but are not limited to, artificial beef flavors, flavors derived from plant proteins such as soy protein to which artificial flavoring has been added (e.g. soy-derived bacon flavoring), and flavors derived from plant proteins such as soy protein with no artificial flavoring.

Artificial beef flavors may be obtained from a variety of sources including Pharma Chemie Inc., TetraGenx, Givaudan S. A., Firmenich, Kemin Industries, Inc., International Flavors & Fragrances Inc., among others.

In another embodiment, the flavoring component include, but is not limited to, strawberry flavor, tutti fruity flavor, orange flavor, banana flavor, mint flavor, and an apple-molasses.

For administration to cattle, sheep, horses and other grazing animals, as well as small animals such as rabbits, hamsters, gerbils, and guinea pigs, grains and seeds are especially appealing additional flavoring agents. The grains may be present in any form consistent with the production of the chew including flour, bran, cereal, fiber, whole grain and meal forms, including gluten meals, and may be rolled, crimped, ground, dehydrated or milled. Minerals may also be added as flavorings, such as salt and other spices. In one embodiment, the grain utilized is dehydrated, milled or flaked. Vegetables such as dehydrated carrots and seeds such as safflower seeds or milo seeds are especially appealing to small animals and may be included. Additionally, flavors such as Sweet Apple and Molasses Flavor Base and others produced by Pharma Chemie, Givaudan S. A. or other suppliers may be utilized in the compositions.

The compositions of the invention may include one or more flavoring agents in an amount that provides the desired level of palatability to the target animal. The one or more flavoring agents will typically be present in a concentration of about 5% to about 40% (w/w). More typically, the flavoring agents will be present in a concentration of about 10% to about 30%, or about 15% to about 25% (w/w).

In one embodiment, the soft chewable compositions of the invention comprise one or more solvents described above, one or more fillers described above, one or more binders described above, one or more humectants described above, one or more surfactants described above, one or more flavors described above, one or more lubricants described above, and optionally one or more disintegrants described above, one or more preservatives described above, one or more stabilizers described above, one or more antioxidants described above and one or more pH modifying agents described above.

In another embodiment, the compositions may comprise one or more solvents selected from various grades of liquid polyethylene glycol (PEG) including PEG 200, PEG 300, PEG 400 and PEG 540; propylene carbonate, propylene glycol; triglycerides including, but not limited to caprylic/capric triglyceride, caprylic/capric/linoleic triglyceride, caprylic/capric/succinic triglyceride, propylene glycol dicaprylate/dicaprate, glycerol caprylate/caprate and polyglycolized glycerides, or a combination thereof; one or more fillers selected from corn starch, pre-gelatinized corn starch, soy protein fines, corn cob, and corn and gluten meal, or a combination thereof; one or more flavors selected from natural and/or artificial pork, beef, fish or poultry flavor, or a combination thereof; one or more binders selected from polyvinylpyrrolidone (e.g. Povidone), cross-linked polyvinylpyrrolidone (Crospovidone), polyethylene glycols of various grades including PEG 3350, PEG 4000, PEG 6000, PEG 8000 and PEG 20,000; and co-polymers of vinylpyrrolidone and vinyl acetate (e.g. Copovidone), or a combination thereof; and one or more surfactants selected from glyceryl monooleate, polyoxyethylene sorbitan fatty acid esters, sorbitan esters including sorbitan monooleate, polyvinyl alcohol, polysorbates including polysorbate 20 and polysorbate 80, d-α-tocopheryl polyethylene glycol 1000 succinate, sodium lauryl sulfate, co-polymers of ethylene oxide and propylene oxide, polyethylene glycol castor oil derivatives including polyoxyl 35 castor oil, polyoxyl 40 hydrogenated castor oil and polyoxyl 60 hydrogenated castor oil; propylene glycol monolaurate; glyceride esters including glycerol caprylate/caprate, polyglycolized glycerides, PEG 300 caprylic/capric glycerides, PEG 400 caprylic/capric glycerides, PEG 300 oleic glycerides, PEG 300 linoleic glycerides; polyethylene glycol stearates and polyethylene glycol hydroxy stearates including polyoxyl 8 stearate, polyoxyl 40 stearate and polyethylene glycol 15 12-hydroxystearate, or a combination thereof; and optionally one or more humectants described above, one or more lubricants described above, one or more preservatives describe above, one or more stabilizers described above, one or more antioxidants described above and one or more pH modifiers described above.

In another embodiment, the compositions comprise one or more solvents selected from various grades of liquid polyethylene glycol including PEG 300, PEG 400 and PEG 540; propylene carbonate; propylene glycol; caprylic/capric triglyceride, caprylic/capric/linoleic triglyceride, propylene glycol dicaprylate/dicaprate and glycerol caprylate/caprate, or a combination thereof; one or more fillers selected from corn starch, pre-gelatinized corn starch, soy protein fines, or a combination thereof; one or more flavors selected from natural and/or artificial pork, beef, fish or poultry flavor, or a combination thereof; one or more binders selected from polyvinylpyrrolidone, cross-linked polyvinylpyrrolidone, polyethylene glycols of various grades including PEG 3350, PEG 4000, PEG 6000 and PEG 8000; and co-polymers of vinylpyrrolidone and vinyl acetate, or a combination thereof; one or more humectants selected from glycerol, propylene glycol, cetyl alcohol and glycerol monostearate, or a combination thereof; and one or more surfactants selected from polyoxyethylene sorbitan fatty acid esters, sorbitan esters including sorbitan monooleate, polysorbates including polysorbate 20 and polysorbate 80, co-polymers of ethylene oxide and propylene oxide, polyethylene glycol castor oil derivatives including polyoxyl 35 castor oil, polyoxyl 40 hydrogenated castor oil and polyoxyl 60 hydrogenated castor oil; propylene glycol monolaurate; PEG 300 caprylic/capric glycerides, PEG 400 caprylic/capric glycerides, PEG 300 oleic glycerides, PEG 300 linoleic glycerides; polyethylene glycol stearates and polyethylene glycol hydroxy stearates including polyoxyl 8 stearate, polyoxyl 40 stearate and polyethylene glycol 15 12-hydroxystearate, or a combination thereof; and optionally one or more lubricants described above, one or more preservatives described above, one or more stabilizers described above, one or more antioxidants described above and one or more pH modifiers described above.

In yet another embodiment, the soft chewable compositions of the invention comprise one or more solvents selected from liquid polyethylene glycols including PEG 200, PEG 300 and PEG 400; caprylic/capric triglyceride and propylene glycol dicaprylate/dicaprate, or a combination thereof; one or more fillers selected from corn starch, pre-gelatinized corn starch and soy protein fines, or a combination thereof; one or more flavors selected from natural and/or artificial beef, fish or poultry flavor, or a combination thereof; one or more binders selected from polyvinylpyrrolidone, cross-linked polyvinylpyrrolidone, polyethylene glycols of various grades including PEG 3350, PEG 4000 and PEG 6000; and co-polymers of vinylpyrrolidone and vinyl acetate, or a combination thereof; one or more humectants selected from glycerol, propylene glycol and cetyl alcohol, or a combination thereof; and one or more surfactants selected from polyoxyethylene sorbitan fatty acid esters, sorbitan esters including sorbitan monooleate, polysorbates including polysorbate 20 and polysorbate 80, polyethylene glycol castor oil derivatives including polyoxyl 35 castor oil, polyoxyl 40 hydrogenated castor oil and polyoxyl 60 hydrogenated castor oil; PEG 300 caprylic/capric glycerides, PEG 400 caprylic/capric glycerides and polyethylene glycol stearates and polyethylene glycol hydroxy stearates including polyoxyl 8 stearate, polyoxyl 40 stearate and polyethylene glycol 15 12-hydroxystearate, or a combination thereof; one or more lubricants selected from polyethylene glycols of various molecular weight ranges including PEG 3350 and PEG 4000, hydrogenated vegetable oils, castor oil, a medium chain triglyceride including caprylic/capric triglycerides and propylene glycol fatty acid esters, or a combination thereof; and optionally one or more preservatives described above, one or more stabilizers described above, one or more antioxidants described above and one or more pH modifiers described above.

In another embodiment, the soft chewable compositions of the invention comprise one or more solvents selected from liquid polyethylene glycols including PEG 300 and PEG 400; caprylic/capric triglyceride and propylene glycol dicaprylate/dicaprate, or a combination thereof; one or more fillers selected from corn starch, pre-gelatinized corn starch and soy protein fines, or a combination thereof; one or more flavors selected from natural and/or artificial beef, fish or poultry flavor, or a combination thereof; one or more binders selected from polyvinylpyrrolidone and polyethylene glycols of various grades including PEG 3350, PEG 4000 and PEG 6000, or a combination thereof; one or more humectants selected from glycerol, propylene glycol and cetyl alcohol, or a combination thereof; and one or more surfactants selected from sorbitan esters including sorbitan monooleate, polysorbates including polysorbate 20 and polysorbate 80, polyethylene glycol castor oil derivatives including polyoxyl 40 hydrogenated castor oil and polyoxyl 60 hydrogenated castor oil; PEG 400 caprylic/capric glycerides and polyethylene glycol stearates and polyethylene glycol hydroxy stearates including polyoxyl 8 stearate, polyoxyl 40 stearate and polyethylene glycol 15 12-hydroxystearate, or a combination thereof; one or more lubricants selected from polyethylene glycols of various molecular weight ranges including PEG 3350 and PEG 4000, caprylic/capric triglycerides and propylene glycol fatty acid esters, or a combination thereof; and optionally one or more preservatives described above, one or more stabilizers described above, one or more antioxidants described above and one or more pH modifiers described above.

In still another embodiment, the soft chewable compositions of the invention comprise one or more solvents selected from liquid polyethylene glycols including PEG 300 and PEG 400; caprylic/capric triglyceride and propylene glycol dicaprylate/dicaprate, or a combination thereof, at a concentration of about 1-20% (w/w) or about 5-20% (w/w); one or more fillers selected from corn starch, pre-gelatinized corn starch and soy protein fines, or a combination thereof, at a concentration of about 30-60% (w/w) or about 30-50% (w/w); one or more flavors selected from natural and/or artificial beef, fish or poultry flavor, or a combination thereof, at a concentration of about 10-30% (w/w) or about 15-25% (w/w); one or more binders selected from polyvinylpyrrolidone and polyethylene glycols of various grades including PEG 3350, PEG 4000 and PEG 6000, or a combination thereof, at a concentration of about 1-10% (w/w) or about 5-15% (w/w); one or more humectants selected from glycerol and propylene glycol, or a combination thereof, at a concentration of about 1-15% (w/w) or about 5-15% (w/w); and one or more surfactants selected from sorbitan esters including sorbitan monooleate, polysorbates including polysorbate 20 and polysorbate 80, polyethylene glycol castor oil derivatives including polyoxyl 40 hydrogenated castor oil and polyoxyl 60 hydrogenated castor oil; PEG 400 caprylic/capric glycerides and polyethylene glycol stearates and polyethylene glycol hydroxy stearates including polyoxyl 8 stearate, polyoxyl 40 stearate and polyethylene glycol 15 12-hydroxystearate, or a combination thereof, at a concentration of about 1-5% (w/w) or about 5-10% (w/w); one or more lubricants selected from polyethylene glycols of various molecular weight ranges including PEG 3350 and PEG 4000, caprylic/capric triglycerides and propylene glycol fatty acid esters, or a combination thereof, at a concentration of about 1-10% (w/w) or about 1-5% (w/w); and optionally one or more preservatives described above, one or more stabilizers described above, one or more antioxidants described above and one or more pH modifiers described above.

In another embodiment, the soft chewable compositions of the invention comprise one or more solvents selected from liquid polyethylene glycols including PEG 300 and PEG 400; and caprylic/capric triglyceride, or a combination thereof, at a concentration of about 5-20% (w/w); one or more fillers selected from corn starch, pre-gelatinized corn starch and soy protein fines, or a combination thereof, at a concentration of about 30-50% (w/w); one or more flavors selected from natural and/or artificial beef, fish or poultry flavor, or a combination thereof, at a concentration of about 15-25% (w/w); one or more binders selected from polyvinylpyrrolidone and polyethylene glycols of various grades including PEG 3350, PEG 4000 and PEG 6000, or a combination thereof, at a concentration of about 5-15% (w/w); one or more humectants selected from glycerol and propylene glycol, or a combination thereof, at a concentration of about 5-15% (w/w); and one or more surfactants selected from polysorbates including polysorbate 20 and polysorbate 80, polyethylene glycol castor oil derivatives including polyoxyl 40 hydrogenated castor oil and polyoxyl 60 hydrogenated castor oil; PEG 400 caprylic/capric glycerides and polyethylene glycol stearates and polyethylene glycol hydroxy stearates including polyoxyl 8 stearate, polyoxyl 40 stearate and polyethylene glycol 15 12-hydroxystearate, or a combination thereof, at a concentration of about 1-5% (w/w) or about 5-10% (w/w); one or more lubricants selected from polyethylene glycols of various molecular weight ranges including PEG 3350 and PEG 4000 and caprylic/capric triglycerides, or a combination thereof at a concentration of about 1-5% (w/w); and optionally one or more preservatives described above, one or more stabilizers described above, one or more antioxidants described above and one or more pH modifiers described above.

In another embodiment, the oral veterinary compositions of the invention are in the form of a chewable tablet. The tablet compositions will comprise an effective amount of at least one systemically-acting active agent described herein, and typically a flavor, a filler, a lubricant, and a flow aid. Optionally, the inventive tablets may further contain at least one of the following ingredients: colorants, binders, antioxidants, disintegrants, or preservatives. Moreover, in an alternative embodiment the invention provides for tablets which are coated. The inventive tablets are prepared according to methods conventional in the art, such as wet and dry granulation processes.

Many of the ingredients for the tablet include those provided for in the soft chewable formulations described above. With respect to fillers (or diluents), the inventive tablets contemplate all the fillers which are known in the tablet art. Non-limiting examples of fillers include anhydrous lactose, hydrated lactose, sprayed dried lactose, crystalline maltose and maltodextrins.

Flow aids or glidants are also well known in the art and include, for example, silicon dioxide (CARBOSIL) or silica gel (SYLOID), talc, starch, calcium, stearate, magnesium stearate, and aluminum magnesium silicate (NEUSILIN). Amounts of flow aids are readily determined by a practitioner in this art and include for using about 0.01 to about 25%, based upon weight of total composition. Non-limiting examples of lubricants for the tablets include magnesium and calcium stearate and stearic acid. Again, the various lubricants are well known to a practitioner of this art as well as the amounts of these compounds. Ranges include from about 0.01 to about 20% (w/w).

In various embodiments, the oral compositions of the invention may be coated. Any suitable coating may be used. In an embodiment, a coating is chosen that will not interfere with an additive. In another embodiment, an additive is chosen that can modify the time for digestion of the additive(s), thereby at least partially controlling the release of the additive(s). Suitable coatings include, but are not limited to, and may be any pharmaceutically acceptable, and/or neutraceutically acceptable coating, as is common in the art. (polymers, monomers). Reference can be had to U.S. Pat. No. 6,498,153, incorporated herein by reference, to Cady et al. for a list of polymers that can function as coatings.

In other embodiments, coatings for the oral veterinary formulations include gelatin, glyceryl behenate, cocoa butter, and beeswax. Other coatings would be known to a practitioner in this art. Coatings for tablets include sugar coatings, such as seal coatings, subcoatings, and syrup coatings, as well as film coatings, such as pan-pour coatings and pan spray coatings. As well known to a practitioner of this art, the coatings contain additional components such as solvents, plasticizers, colorants, opaquant-extenders and film formers.

### Method of Manufacture

The soft chews of the invention are prepared by mixing the active ingredient(s) with the non-active excipients in a mixer and mixing the components to achieve a dough-like mixture wherein the active ingredient(s) are homogeneously distributed. The resulting dough-like mixture is then formed into soft chewable dosage units of different sizes for different size animals.

In one embodiment, the process to manufacture the soft chews will not include the addition of water, although there may be some amount of water included with certain components used. The presence of significant amounts of water in veterinary compositions is known to affect the stability of certain active agents. Thus, in certain embodiments of the invention water will not be added to the composition where active agents and/or excipients are used that are susceptible to degradation in the presence of water.

The temperature at which the soft chewable veterinary compositions of the invention are prepared is dependent on the stability requirements of the active and non-active components of the compositions. In certain cases where ingredients that are not temperature-sensitive are used, higher processing temperatures may be tolerable. However, when active and non-active ingredients are used that are sensitive to temperature, the process may be adapted to operate at a temperature range that will not adversely impact the stability of the composition. In some embodiments, the process will preferably not impart significant amounts of heat during any one processing step to avoid the possible degradation of any of the components of the composition. Thus, in some embodiments, any one step of the process may be operated so that the average temperature of the mixture does not rise more than about 20° C. above room temperature (room temperature will be considered 20-25° C.). In other embodiments, the process will be conducted so that the average temperature of the mixture does not rise more than about 15° C., more than about 10° C. or more than about 5° C. above room temperature. In still another embodiment, the process may be conducted so that the average temperature of the mixture will not rise more than about 3° C. above room temperature. In some embodiments, the required temperature may be maintained by the use of process cooling devices. In other embodiments, the required temperature may be maintained by using equipment that does not produce sufficient heat to maintain the required temperature of the mixture during processing.

In one embodiment, active and inactive ingredients for the soft chews of the invention are added to a mixing vessel such as a planetary or double planetary mixer or a horizontal mixer capable of blending the material and casting it against the side of the mixing vessels. This action permits the ingredients to be well and consistently blended without application of heat or addition of pharmaceutical grade water to the mixture.

Horizontal mixers generally comprise a mixing chamber, an elongated, horizontal mixing shaft which rotates, and a plurality of mixing tools which depend generally perpendicularly from the horizontal shaft to rotate around the inside of the chamber (see, e.g., U.S. Pat. No. 5,735,603, the disclosure of which is incorporated herein by this reference). The mixing tools are configured and dimensioned as required for the mixing process to follow the shape of the chamber walls as rotated for proper mixing of all of material present. Some such mixing chambers are cylindrically shaped, while others are trough-shaped, such as mixers which are commonly referred to in the art as double-arm mixers or ribbon mixers.

In one embodiment, the soft chewable compositions of the invention may be formed from the dough-like mixture by any suitable forming techniques known in the art including forming by hand. One of skill in the art will understand that once the homogeneous dough mixture having the required properties is prepared the individual dosage units of various sizes may be formed by weighing the required amount of the dough-like mixture and forming the soft chewable compositions by hand or using any other molding techniques known in the art. In one embodiment, the dough-like mixture is extruded to form the soft chewable dosage forms. In another embodiment, the soft chewable dosage forms are formed using a forming machine. A variety of forming equipment may be utilized in the invention including molding machines developed for use in producing molded food products, such as pre-formed hamburger patties and chicken nuggets. For example, the molding machines described in U.S. Pat. Nos. 3,486,186; 3,887,964; 3,952,478; 4,054,967; 4,097,961; 4,182,003; 4,334,339; 4,338,702; 4,343,068; 4,356,595; 4,372,008; 4,523,520; 4,535,505; 4,597,135; 4,608,731; 4,622,717; 4,697,308; 4,768,941; 4,780,931; 4,818,446; 4,821,376; 4,872,241; 4,975,039; 4,996,743; 5,021,025; 5,022,888; 5,165,218; 5,655,436; 5,980,228 and 7,780,931 (the disclosures of which are incorporated herein by reference) are representative of forming equipment that may be utilized in the invention.

In one embodiment forming equipment that does not apply compression heat to the chew mixture may be utilized. Non-limiting examples of forming machines include those manufactured by NuTec Manufacturing including model nos. 710, 720, 745, 750 and 760; and those manufactured by the Formax Corporation, including the VerTex 1000, NovaMax 500, Maxum 700, Ultra 26, F-19, F-400 and F-6. The order of mixing the components is not critical and various processing schemes may be used to form the dough-like mixture prior to forming the soft chew dosage units. In some embodiments, the active ingredient(s) and possibly some non-active components such as preservatives or antioxidants may first be dissolved in a solvent(s) prior to mixing with other non-active components of the composition in a blender to form a dough-like mixture. The liquid components may be added at a controlled rate to ensure homogeneity of the mixture. Alternatively, the active ingredient(s) may be mixed in dry form (solid state) with other non-active components in a blender and liquid components may be added to the dry blended mixture with further mixing to form a uniform dough-like mixture. In still another embodiment, the liquid components of the invention may first be placed in the blender and the dry components, including active agent(s) may be added to the liquid with further mixing to form a uniform dough-like mixture.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may be best understood in conjunction with the accompanying drawings, in which:

FIG. 1 is a schematic representation of IgY production;

FIG. 2 shows a putative mode of action of IgY-mediated protection against “bacterial species” associated diarrhea;

FIG. 3 is a partial section, taken in a horizontal plane, through a coated tooth, showing the irregular tooth surface, the conformation of the coating to the tooth surface and its relative thickness, all on a much enlarged scale. The hydrophobic barrier film, containing antimicrobial IgY and other functional agents, conforms to the substrate and fills pits, fissures, cracks and other irregularities of the tooth surface. The transfer layer facilitates adhesion of the hydrophobic barrier film to the tooth surface;

FIGS. 4A and 4B are enlarged views of the coated tooth surface, showing the area of the tooth surface in FIG. 3, to demonstrate the electrostatic charge distribution at the interface between the tooth surface and the transfer agent. These figures illustrate the mode of attachment of the transfer agent to the negatively charged tooth surface. (A) The molecules of the positively charged surfactant form a dense monolayer which attaches to the negatively charged substrate. The alkyl groups of the transfer agent face away from the surface. (B) Polyamine molecules adsorb to the substrate with their hydrophobic side groups facing away from the hydrophilic tooth surface;

FIG. 5A shows the structure of gingipain precursor polypeptides RgpB, RgpA, Kgp and HagA;

FIG. 5B shows the processed RgpA, Kgp and HagA polyproteins.

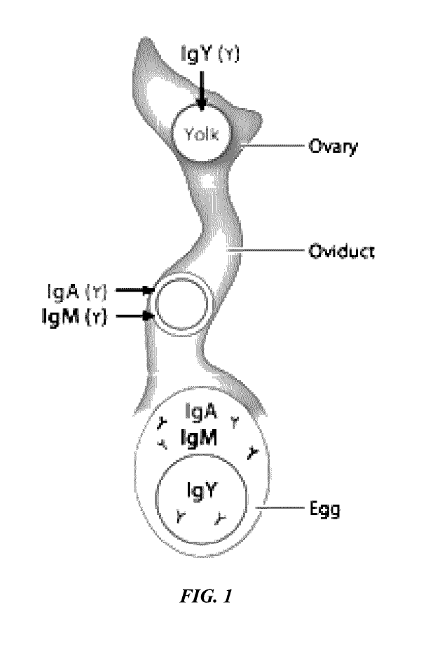

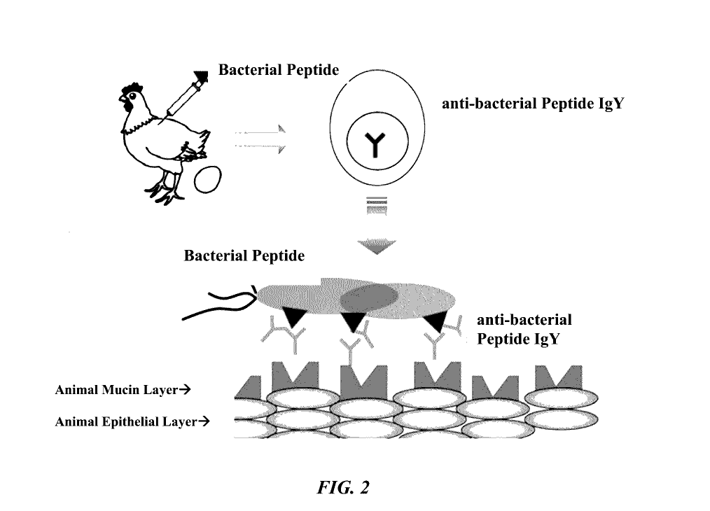

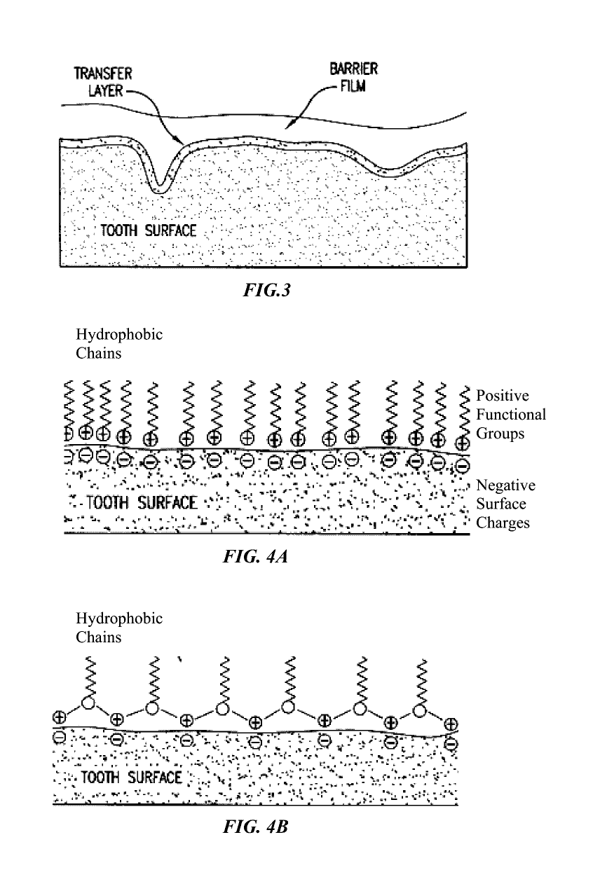

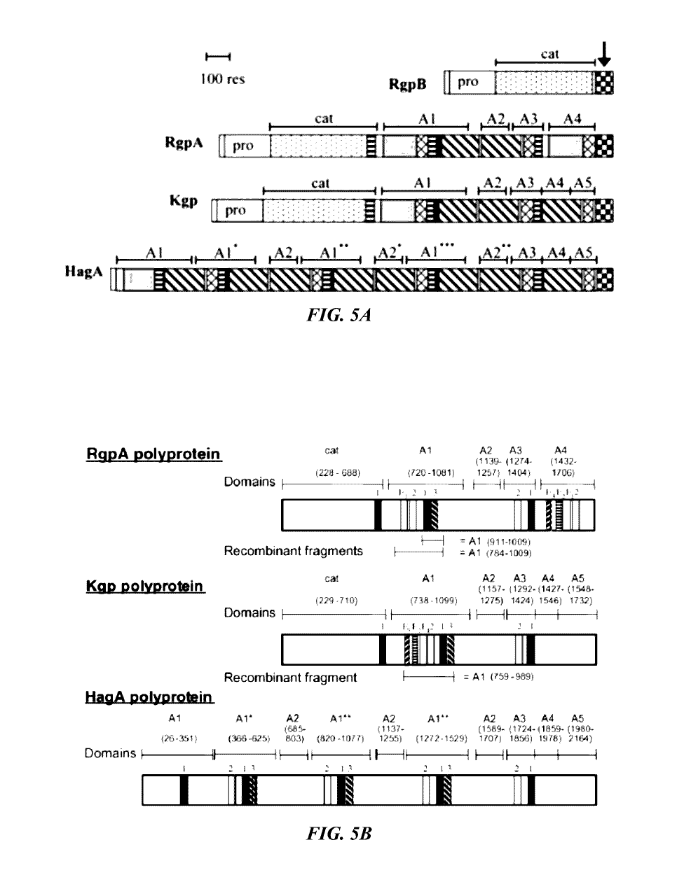

### DETAILED DESCRIPTION

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

By “animal” is intended mammals, human, birds, and the like. The animal may be selected from equine (e.g., horse), canine (e.g., dogs, wolves, foxes, coyotes, jackals), feline (e.g., lions, tigers, domestic cats, wild cats, other big cats, and other feline including cheetahs and lynx), ovine (e.g., sheep), bovine (e.g., cattle, cow, buffalo), swine (pig), avian (e.g., chicken, duck, goose, turkey, quail, pheasant, parrot, finches, hawk, crow, ostrich, emu and cassowary), primate (e.g., prosimian, tarsier, monkey, gibbon, ape), and fish. The term “animal” also includes an individual animal in all stages of development, including embryonic and fetal stages.

As used herein, the term “antigen” or “immunogen” means a substance that induces a specific immune response in a host animal. The antigen may comprise a whole organism, killed, attenuated or live; a subunit or portion of an organism; a recombinant vector containing an insert expressing an epitope, polypeptide, peptide, protein, or fragment thereof with immunogenic properties; a piece or fragment of nucleic acid capable of inducing an immune response upon presentation to a host animal; a protein, a polypeptide, a peptide, an epitope, a hapten, or any combination thereof. Alternately, the immunogen or antigen may comprise a toxin or antitoxin.

The term “immunogenic protein or peptide” as used herein also includes peptides and polypeptides that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. The term epitope, also known as antigenic determinant, is the part of a macromolecule recognized by the immune system and able to induce an immune reaction of the humoral type (B cells) and/or cellular type (T cells).

The term “immunogenic protein or peptide” further contemplates deletions, additions and substitutions to the sequence, so long as the polypeptide functions to produce an immunological response as defined herein. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide.

The term epitope is the part of a macromolecule recognized by the immune system and able to induce an immune reaction of the humoral type (B cells) and/or cellular type (T cells). The term is also used interchangeably with “antigenic determinant” or “antigenic determinant site”. Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

The term “immunogenic” protein or polypeptide as used herein also refers to an amino acid sequence which elicits an immunological response as described above. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996). For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., 1984; Geysen et al., 1986. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

Synthetic antigens are also included within the definition, for example, polyepitopes, flanking epitopes, and other recombinant or synthetically derived antigens. Immunogenic fragments, for purposes of the present invention, will usually include at least about 3 amino acids, about 5 amino acids, about 10-15 amino acids, about 15-25 amino acids or more amino acids, of the molecule. There is no critical upper limit to the length of the fragment, which could comprise nearly the full-length of the protein sequence, or even a fusion protein comprising at least one epitope of the protein.

Accordingly, a minimum structure of a polynucleotide expressing an epitope is that it comprises or consists essentially of or consists of nucleotides to encode an epitope or antigenic determinant of protein or polypeptide from or derived from a microorganism that causes dental or oral health disease. A polynucleotide encoding a fragment of the total protein or polypeptide comprises or consists essentially of or consists of a minimum of 15 nucleotides, advantageously about 30-45 nucleotides, and preferably about 45-75, at least 57, 87 or 150 consecutive or contiguous nucleotides of the sequence encoding the total protein or polypeptide. Epitope determination procedures, such as, generating overlapping peptide libraries (Hemmer et al., 1998), Pepscan (Geysen et al., 1984; Geysen et al., 1985; Van der Zee R. et al., 1989; Geysen, 1990; Multipin® Peptide Synthesis Kits de Chiron) and algorithms (De Groot et al., 1999), can be used in the practice of the invention, without undue experimentation.

A “polynucleotide” is a polymeric form of nucleotides of any length that contains deoxyribonucleotides, ribonucleotides, and analogs in any combination. Polynucleotides may have three-dimensional structure, and may perform any function, known or unknown. The term “polynucleotide” includes double-, single-, and triple-stranded helical molecules. Unless otherwise specified or required, any embodiment of the invention described herein that is a polynucleotide encompasses both the double stranded form and each of two complementary forms known or predicted to make up the double stranded form of either the DNA, RNA or hybrid molecule.

The term “codon optimization” refers to the process of optimally configuring the nucleic acid sequence encoding a protein, polypeptide, antigen, epitope, domain or fragment for expression/translation in a selected host. In general, gene expression levels depend on many factors, such as promoter sequences and regulatory elements. One of the most important factors is the adaptation of the codon usage of the transcript gene to the typical codon usage of the host (Lithwich, G. and Margalit, H., Genome Res. 13, 2665-2673, 2003). Therefore, highly expressed genes in prokaryotic genomes under translational selection have a pronounced codon usage bias. This is because they use a small subset of codons that are recognized by the most abundant tRNA species (Ikemura, T., J. Mol. Biol. 151, 389-409, 1981). The force that modulates this codon adaptation is called translational selection and its strength is important in fast-growing bacteria (Rocha, E. P., Genome Res. 14, 2279-2286, 2004; Sharp, P. M. et al., Nucleic Acids Res. 33, 1141-1153). If a gene contains codons that are rarely used by the host, its expression level will not be maximal. This may be one of the limitations of heterologous protein expression (Gustafsson, C. et al., Trends Biotechnol. 22, 346-353, 2004) and the development of DNA vaccines (Ivory, C. and Chadee, K., Genet. Vaccines Ther. 2, 17, 2004). A high number of synthetic genes have been re-designed to increase their expression level. The Synthetic Gene Database (SGDB) (Wu, G. et al., Nucleic Acids Res. 35, D76-D79, 2007) contains information from more than 200 published experiments on synthetic genes. In the design process of a nucleic acid sequence that will be inserted into a new host to express a certain protein in optimal amounts, codon usage optimization is usually one of the first steps (Gustafsson, C., Trends Biotechnol. 22, 346-353, 2004). Codon usage optimization basically involves altering the rare codons in the target gene so that they more closely reflect the codon usage of the host without modifying the amino acid sequence of the encoded protein (Gustafsson, C., Trends Biotechnol. 22, 346-353, 2004). The information usually used for the optimization process is therefore the DNA or protein sequence to be optimized and a codon usage table (reference set) of the host.

The following are non-limiting examples of polynucleotides: a gene or gene fragment, exons, introns, mRNA, tRNA, rRNA, siRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The invention further comprises a complementary strand to a polynucleotide encoding a herpesvirus protein, antigen, epitope or immunogen. The complementary strand can be polymeric and of any length, and can contain deoxyribonucleotides, ribonucleotides, and analogs in any combination thereof

The terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

An “isolated” polynucleotide or polypeptide is one that is substantially free of the materials with which it is associated in its native environment. By substantially free, is meant at least 50%, at least 70%, at least 80%, at least 90%, or at least 95% free of these materials.

Hybridization reactions can be performed under conditions of different stringency. Conditions that increase stringency of a hybridization reaction are well known. See for example, “Molecular Cloning: A Laboratory Manual”, second edition (Sambrook et al., 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The invention further encompasses polynucleotides encoding functionally equivalent variants and derivatives of the oral health compromising (OHC) polypeptides and functionally equivalent fragments thereof that may enhance, decrease or not significantly affect inherent properties of the polypeptides encoded thereby. These functionally equivalent variants, derivatives, and fragments display the ability to retain the activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide.

In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the OHC polynucleotide or polypeptide of interest.

### EXAMPLES

The invention is further described by the following non-limiting examples which further illustrate the invention, and are not intended, nor should they be interpreted to, limit the scope of the invention.

Soft chews containing the Antimicrobial IgY antibody alone and in combination with a macrocyclic lactone were prepared with a variety of non-active excipients and evaluated for effectiveness to control endoparasites and ectoparasites in cats and dogs. In addition, soft chewable compositions comprising one or more parasiticides that are active against endoparasites were prepared and evaluated for efficacy against various internal parasites.

### Example 1

### Preparation of Soft Chewable Veterinary Formulations

The soft chewable formulation of Table 1 was prepared by the following procedure: the active agent(s) and potassium sorbate (if present) were dissolved in the corresponding amount of solvent by mixing at ambient temperature. In a blender, the filler (e.g. soy protein fines and/or starch) are mixed together at ambient temperature until blended, then the other non-active components and the pre-made solution of the active agent(s) and potassium sorbate (if present) are added to the mixture. The mixture is stirred further until a well-blended dough-type mixture is formed.

The dough-like mixture is then formed into individual soft chewable dosage units in nominal sizes of 0.5 g, 1 g and 4 g. The formulations in Tables 2-24 may be prepared by similar procedures. In the tables below, the abbreviation “QS” meaning “quantum sufficit” is intended to mean that the amount of corresponding component may be adjusted to bring the composition to 100% (w/w).

TABLE 1

| Ingredients | Function | % (w/w) |
|---|---|---|
| Antimicrobial IgY Antibody | Active | 2.2 |
| Soy Protein Fines | Filler | 26.5 (QS) |
| Corn Starch | Filler | 31.0 |
| artificial meat flavor | Flavoring | 5.1 |
| artificial beef flavor | Flavoring | 7.1 |
| Povidone K-30 | Binder | 2.8 |
| PEG 400 | Solvent | 7.1 |
| PEG 4000 | Binder | 6.4 |
| polyethylene glycol 12-hydroxystearate | Surfactant | 3.0 |
| Glycerin | Humectant | 5.1 |
| Potassium Sorbate | Preservative | 0.3 |
| Caprylic/capric triglyceride | Solvent/Lubricant | 3.2 |

TABLE 2

| Ingredients | Function | % (w/w) |
|---|---|---|
| Antimicrobial IgY Antibody | Active | 2.2 |
| Soy Protein Fines | Filler | 56.0 (QS) |
| artificial meat flavor | Flavoring | 5.5 |
| artificial beef flavor | Flavoring | 7.5 |
| Povidone K-30 | Binder | 2.8 |
| PEG 4000 | Binder | 6.4 |
| Sorbitan monooleate | Surfactant | 4.0 |
| Glycerin | Humectant | 5.1 |
| Potassium Sorbate | Preservative | 0.3 |
| Propylene glycol dicaprylate/dicaprate | Solvent/Lubricant | 3.2 |
| Propylene glycol | Solvent | 7.0 |

TABLE 3

| Ingredients | Function | % (w/w) |
|---|---|---|
| Antimicrobial IgY Antibody | Active | 2.2 |
| Soy Protein Fines | Filler | 31 (QS) |
| Corn Gluten Meal | Filler | 30.0 |
| artificial beef flavor | Flavoring | 12.0 |
| Povidone K-30 | Binder | 2.8 |
| PEG 4000 | Binder | 6.4 |
| Polyoxyl 60 hydrogenated castor oil | Surfactant | 4.0 |
| Potassium Sorbate | Preservative | 0.3 |
| Propylene glycol dicaprylate/dicaprate | Solvent/Lubricant | 3.2 |
| PEG 400 | Solvent | 8.0 |

TABLE 4

| Ingredients | Function | % (w/w) |
|---|---|---|
| Antimicrobial IgY Antibody | Active | 2.2 |
| Soy Protein Fines | Filler | 32.0 (QS) |
| Pre-gelatinized Corn Starch | Filler | 31.0 |
| artificial beef flavor | Flavoring | 12.0 |
| Povidone K-30 | Binder | 2.8 |
| PEG 4000 | Binder | 6.4 |
| polyoxyl 35 castor oil | Surfactant | 4.0 |
| Potassium Sorbate | Preservative | 0.3 |
| Propylene glycol dicaprylate/dicaprate | Solvent/Lubricant | 3.2 |
| PEG 400 | Solvent | 6.0 |

TABLE 5

| Ingredients | Function | % (w/w) |
|---|---|---|
| Antimicrobial IgY Antibody | Active | 2.2 |
| Soy Protein Fines | Filler | 26.0 (QS) |
| Pre-gelatinized Corn Starch | Filler | 30.0 |
| beef flavor | Flavoring | 15.0 |
| Copovidone | Binder | 3.3 |
| PEG 4000 | Binder | 5.5 |
| Polyoxyl 60 hydrogenated castor oil | Surfactant | 4.0 |
| Glycerin | Humectant | 5.1 |
| Potassium Sorbate | Preservative | 0.3 |
| Propylene glycol dicaprylate/dicaprate | Solvent/Lubricant | 3.2 |
| PEG 400 | Solvent | 5.4 |

TABLE 6

| Ingredients | Function | % (w/w) |
|---|---|---|
| Antimicrobial IgY Antibody | Active | 1.5 |
| Soy Protein Fines | Filler | 46.5 (QS) |
| beef flavor | Flavoring | 20.0 |
| Povidone K-30 | Binder | 7.0 |
| PEG 400 | Solvent | 15 |
| polyethylene glycol 12-hydroxystearate | Surfactant | 3.0 |
| Caprylic/capric triglyceride | Solvent/lubricant | 7.0 |

TABLE 7

| Ingredients | Function | % (w/w) |
|---|---|---|
| Antimicrobial IgY Antibody | Active | 1.875 |
| Soy Protein Fines | Filler | 46.1 (QS) |
| beef flavor | Flavoring | 20.0 |
| Povidone K-30 | Binder | 8.5 |
| PEG 400 | Solvent | 15.5 |
| polyethylene glycol 12-hydroxystearate | Surfactant | 3.0 |
| Caprylic/capric triglyceride | Solvent/lubricant | 5.0 |

TABLE 8

| Ingredients | Function | % (w/w) |
|---|---|---|
| Antimicrobial IgY Antibody | Active | 1.875 |
| Soy Protein Fines | Filler | 36.1 (QS) |
| beef flavor | Flavoring | 20.0 |
| Povidone K-30 | Binder | 8.5 |
| PEG 400 | Solvent | 15.5 |
| polyethylene glycol 12-hydroxystearate | Surfactant | 3.0 |
| Caprylic/capric triglyceride | Solvent/lubricant | 5.0 |
| Croscarmellose sodium | disintegrant | 10.0 |

TABLE 9

| Ingredients | Function | % (w/w) |
|---|---|---|
| Antimicrobial IgY Antibody | Active | 2.3 |
| Soy Protein Fines | Filler | 20.6 (QS) |
| Corn Starch | Filler | 25.0 |
| beef flavor | Flavoring | 20.5 |
| Povidone K-30 | Binder | 2.8 |
| PEG 400 | Solvent | 7.2 |
| PEG 4000 | Binder | 6.4 |
| polyethylene glycol 12-hydroxystearate | Surfactant | 3.1 |
| Glycerin | Humectant | 8.6 |
| Potassium Sorbate | Preservative | 0.3 |
| Caprylic/capric triglyceride | Solvent/Lubricant | 3.1 |

TABLE 10

| Ingredients | Function | % (w/w) |
|---|---|---|
| Antimicrobial IgY Antibody | Active | 2.3 |
| Soy Protein Fines | Filler | 20.0 (QS) |
| Corn Starch | Filler | 25.0 |
| beef flavor | Flavoring | 20.0 |
| Povidone K-30 | Binder | 2.8 |
| PEG 400 | Solvent | 7.1 |
| PEG 4000 | Binder | 6.4 |
| polyethylene glycol 12-hydroxystearate | Surfactant | 3.1 |
| Glycerin | Humectant | 10.0 |
| Potassium Sorbate | Preservative | 0.3 |
| Caprylic/capric triglyceride | Solvent/Lubricant | 3.2 |

TABLE 11

| Ingredients | Function | % (w/w) |
|---|---|---|
| Antimicrobial IgY Antibody | Active | 1.89 |
| Milbemycin oxime | Active | 0.375 |
| Soy Protein Fines | Filler | 20.0 (QS) |
| Corn Starch | Filler | 24.7 |
| beef flavor | Flavoring | 20.2 |
| Povidone K-30 | Binder | 2.7 |
| PEG 400 | Solvent | 7.1 |
| PEG 4000 | Binder | 6.3 |
| polyethylene glycol 12-hydroxystearate | Surfactant | 3.0 |
| Glycerin | Humectant | 10.1 |
| Caprylic/capric triglyceride | Solvent/Lubricant | 3.1 |
| Potassium Sorbate | Preservative | 0.3 |
| BHT | Antioxidant | 0.14 |

TABLE 12

| Ingredients | Function | % (w/w) |
|---|---|---|
| Antimicrobial IgY Antibody | Active | 1.875 |
| Milbemycin oxime | Active | 0.375 |
| Soy Protein Fines | Filler | 19.4 (QS) |
| Corn Starch | Filler | 25.0 |
| beef flavor | Flavoring | 20.0 |
| Povidone K-30 | Binder | 2.75 |
| PEG 400 | Solvent | 7.1 |
| PEG 4000 | Binder | 6.35 |
| polyethylene glycol 12-hydroxystearate | Surfactant | 3.1 |
| Glycerin | Humectant | 10.0 |
| Caprylic/capric triglyceride | Solvent/Lubricant | 3.15 |
| Potassium Sorbate | Preservative | 0.3 |
| BHT | Antioxidant | 0.14 |
| Citric Acid Monohydrate | pH modifier | 0.50 |

TABLE 13

| Ingredients | Function | % (w/w) |
|---|---|---|
| Antimicrobial IgY Antibody | Active | 1.875 |
| Milbemycin oxime | Active | 0.375 |
| Soy Protein Fines | Filler | 20.5 (QS) |
| Corn Starch | Filler | 24.0 |
| beef flavor | Flavoring | 20.0 |
| Copovidone | Binder | 2.75 |
| PEG 300 | Solvent | 8.0 |
| PEG 4000 | Binder | 6.35 |
| Polyoxyl 60 hydrogenated castor oil | Surfactant | 3.1 |
| Glycerin | Humectant | 10.0 |
| Propylene glycol dicaprylate/dicaprate | Solvent/Lubricant | 2.15 |
| Potassium Sorbate | Preservative | 0.3 |
| BHT | Antioxidant | 0.14 |
| Citric Acid Monohydrate | pH modifier | 0.50 |

TABLE 14

| Ingredients | Function | % (w/w) |
|---|---|---|
| Antimicrobial IgY Antibody | Active | 1.875 |
| Ivermectin | Active | 0.375 |
| Soy Protein Fines | Filler | 29.4 (QS) |
| Pre-gelatinized corn starch | Filler | 15.0 |
| beef flavor | Flavoring | 20.0 |
| Copovidone | Binder | 2.75 |
| Caprylate/caprate glyceride | Solvent | 8.0 |
| PEG 4000 | Binder | 6.35 |
| polyoxyl 35 castor oil (Cremophor ® EL) | Surfactant | 3.1 |
| Propylene glycol | Humectant | 10.0 |
| Propylene glycol dicaprylate/dicaprate | Solvent/Lubricant | 2.2 |
| Potassium Sorbate | Preservative | 0.3 |
| BHT | Antioxidant | 0.14 |
| Citric Acid Monohydrate | pH modifier | 0.50 |

TABLE 15

| Ingredients | Function | % (w/w) |
|---|---|---|
| Antimicrobial IgY Antibody | Active | 2.2 |
| Moxidectin | Active | 0.50 |
| Soy Protein Fines | Filler | 29.4 (QS) |
| Pre-gelatinized corn starch | Filler | 15.0 |
| beef flavor | Flavoring | 20.0 |
| Povidone K30 | Binder | 2.75 |
| Caprylate/caprate glyceride | Solvent | 8.0 |
| PEG 4000 | Binder | 6.0 |
| Polyoxyl 40 hydrogenated castor oil | Surfactant | 3.1 |
| (Cremophor ® RH40) | | |
| Propylene glycol | Humectant/Solvent | 10.0 |
| Propylene glycol dicaprylate/dicaprate | Solvent/Lubricant | 2.2 |
| Potassium Sorbate | Preservative | 0.3 |
| BHT | Antioxidant | 0.14 |
| Citric Acid Monohydrate | pH modifier | 0.50 |

TABLE 16

| Ingredients | Function | % (w/w) |
|---|---|---|
| Antimicrobial IgY Antibody | Active | 0.5 |
| Soy Protein Fines | Filler | 16.6 |
| Corn Starch | Filler | 32.5 (QS) |
| beef flavor | Flavoring | 19.4 |
| Povidone K-30 | Binder | 2.6 |
| PEG 400 | Solvent | 7.8 |
| PEG 4000 | Binder | 6.1 |
| polyethylene glycol 12-hydroxystearate | Surfactant | 4.7 |
| Lauroyl polyoxyl-32 glycerides | Surfactant | 4.7 |
| Potassium Sorbate | Preservative | 0.3 |
| Caprylic/capric triglyceride | Solvent/Lubricant | 4.9 |

TABLE 17

| Ingredients | Function | % (w/w) |
|---|---|---|
| Antimicrobial IgY Antibody | Active | 0.5 |
| Soy Protein Fines | Filler | 19.4 |
| Pre-gelatinized corn Starch | Filler | 29.7 (QS) |
| beef flavor | Flavoring | 18.0 |
| Copovidone | Binder | 3.0 |
| PEG 540 | Solvent | 8.3 |
| PEG 4000 | Binder | 6.1 |
| Polyoxyl 60 hydrogenated castor oil | Surfactant | 5.1 |
| (Cremophor ® RH60) | | |
| Lauroyl polyoxyl-32 glycerides | Surfactant | 4.7 |
| Potassium Sorbate | Preservative | 0.3 |
| Caprylic/capric triglyceride | Solvent/Lubricant | 4.9 |

TABLE 18

| Ingredients | Function | % (w/w) |
|---|---|---|
| Antimicrobial IgY Antibody | Active | 0.5 |
| Soy Protein Fines | Filler | 26.9 (QS) |
| Corn Starch | Filler | 23.4 |
| beef flavor | Flavoring | 20.0 |
| PEG 400 | Solvent | 6.8 |
| PEG 4000 | Binder | 5.8 |
| polyethylene glycol 12-hydroxystearate | Surfactant | 4.8 |
| Lauroyl polyoxyl-32 glycerides | Surfactant | 6.3 |
| Potassium Sorbate | Preservative | 0.3 |
| Caprylic/capric triglyceride | Solvent/Lubricant | 5.2 |

TABLE 19

| Ingredients | Function | % (w/w) |
|---|---|---|
| Antimicrobial IgY Antibody | Active | 0.5 |
| Soy Protein Fines | Filler | 24.3 (QS) |
| Pre-gelatinized corn starch | Filler | 26.0 |
| beef flavor | Flavoring | 19.0 |
| PEG 540 | Solvent | 6.8 |
| Crospovidone | Binder | 5.8 |
| polyoxyl 35 castor oil (Cremophor ® EL) | Surfactant | 5.2 |
| Lauroyl polyoxyl-32 glycerides | Surfactant | 6.9 |
| Potassium Sorbate | Preservative | 0.3 |
| Caprylic/capric triglyceride | Solvent/Lubricant | 5.2 |

TABLE 20

| Ingredients | Function | % (w/w) |
|---|---|---|
| Antimicrobial IgY Antibody | Active | 0.5 |
| Soy Protein Fines | Filler | 41.6 (QS) |
| beef flavor | Flavoring | 19.9 |
| Povidone K-30 | Binder | 4.6 |
| PEG 400 | Solvent | 15.1 |
| PEG 4000 | Binder | 8.1 |
| polyethylene glycol 12-hydroxystearate | Surfactant | 4.6 |
| Potassium Sorbate | Preservative | 0.3 |
| Caprylic/capric triglyceride | Solvent/Lubricant | 4.6 |

TABLE 21

| Ingredients | Function | % (w/w) |
|---|---|---|
| Antimicrobial IgY Antibody | Active | 0.5 |
| Soy Protein Fines | Filler | 44.2 (QS) |
| beef flavor | Flavoring | 18.0 |
| Povidone K-30 | Binder | 4.6 |
| PEG 400 | Solvent | 15.1 |
| Cross-linked polyvinylpyrrolidone | Binder | 7.1 |
| propylene glycol monolaurate | Surfactant | 4.6 |
| Potassium Sorbate | Preservative | 0.3 |
| Caprylic/capric triglyceride | Solvent/Lubricant | 5.6 |

TABLE 22

| Ingredients | Function | % (w/w) |
|---|---|---|
| Antimicrobial IgY Antibody | Active | 0.5 |
| Corn Starch | Filler | 40.8 (QS) |
| beef flavor | Flavoring | 19.9 |
| Povidone K-30 | Binder | 5.7 |
| PEG 400 | Solvent | 11.4 |
| PEG 4000 | Binder | 5.7 |
| polyethylene glycol 12-hydroxystearate | Surfactant | 2.7 |
| Lauroyl polyoxyl-32 glycerides | Surfactant | 2.7 |
| Potassium Sorbate | Preservative | 0.3 |
| Caprylic/capric triglyceride | Solvent/Lubricant | 5.4 |
| Sodium Starch glycolate | Disintegrate | 5.0 |

TABLE 23

| Ingredients | Function | % (w/w) |
|---|---|---|
| Antimicrobial IgY Antibody | Active | 0.5 |
| Soy Protein Fines | Filler | 19.4 |
| Corn Starch | Filler | 24.0 (QS) |
| beef flavor | Flavoring | 19.2 |
| Povidone K-30 | Binder | 2.6 |
| PEG 400 | Solvent | 8.6 |
| PEG 4000 | Binder | 6.0 |
| polyethylene glycol 12-hydroxystearate | Surfactant | 4.6 |
| Lauroyl polyoxyl-32 glycerides | Surfactant | 4.6 |
| Potassium Sorbate | Preservative | 0.3 |
| Caprylic/capric triglyceride | Solvent/Lubricant | 5.3 |
| Glycerin | Humectant | 4.8 |

TABLE 24

| Ingredients | Function | % (w/w) |
|---|---|---|
| Antimicrobial IgY Antibody | Active | 2.3 |
| Soy Protein Fines | Filler | 22.0 (QS) |
| Corn Starch | Filler | 26.4 |
| beef flavor | Flavoring | 10.0 |
| Artificial Powdered Meat Flavor | Flavoring | 10.0 |
| Povidone K-30 | Binder | 2.7 |
| PEG 400 | Solvent | 7.0 |
| PEG 4000 | Binder | 6.25 |
| polyethylene glycol 12-hydroxystearate | Surfactant | 3.0 |
| Glycerin | Humectant | 7.0 |
| Potassium Sorbate | Preservative | 0.3 |
| Caprylic/capric triglyceride | Solvent/Lubricant | 3.0 |

TABLE 25

| Ingredients | Function | % (w/w) |
|---|---|---|
| Antimicrobial IgY Antibody | Active | 13.6 |
| Soy Protein Fines | Filler | 15-25 (QS) |
| Corn Starch | Filler | 15-25 |
| beef flavor | Flavoring | 20 |
| PEG 400 | Solvent | 11.9 |
| PEG 4000 | Binder | 5 |
| polyethylene glycol 12-hydroxystearate | Surfactant | 3-5 |
| Glycerin | Humectant | 2-5 |
| Potassium Sorbate | Preservative | 0.3 |

TABLE 26

| Ingredients | Function | % (w/w) |
|---|---|---|
| Antimicrobial IgY Antibody | Active | 13.6 |
| Corn Starch | Filler | 41 (QS) |
| beef flavor | Flavoring | 15-25 |
| PEG 400 | Solvent | 11.9 |
| Cross-linked polyvinylpyrrolidone | Binder | 5 |
| polyoxyl 35 castor oil | Surfactant | 3-5 |
| Propylene glycol | Humectant | 2-5 |
| Potassium Sorbate | Preservative | 0.3 |

TABLE 27

| Ingredients | Function | % (w/w) |
|---|---|---|
| Antimicrobial IgY Antibody | Active | 13.6 |
| Soy Protein Fines | Filler | 12.6 |
| Corn Starch | Filler | 25 (QS) |
| beef flavor | Flavoring | 20 |
| Povidone K-30 | Binder | 2.75 |
| PEG 400 | Solvent | 5.5 |
| PEG 4000 | Binder | 6.2 |
| polyethylene glycol 12-hydroxystearate | Surfactant | 5.0 |
| Glycerin | Humectant | 7-8 |
| Potassium Sorbate | Preservative | 0.3 |
| Caprylic/capric triglyceride | Solvent/Lubricant | 2.0 |

TABLE 28

| Ingredients | Function | % (w/w) |
|---|---|---|
| Antimicrobial IgY Antibody | Active | 13.6 |
| Soy protein fines | Filler | 25.0 (QS) |
| Corn Starch | Filler | 15-18 |
| beef flavor | Flavoring | 20 |
| PEG 400 | Solvent | 11.9 |
| Cross-linked polyvinylpyrrolidone | Binder | 5 |
| polyoxyl 35 castor oil | Surfactant | 3-5 |
| Propylene glycol | Humectant | 2-5 |
| Potassium Sorbate | Preservative | 0.3 |

TABLE 29

| Ingredients | Function | % (w/w) |
|---|---|---|
| Antimicrobial IgY Antibody | Active | 13.6 |
| Soy protein fines | Filler | 15.2 (QS) |
| Corn Starch | Filler | 25 |
| beef flavor | Flavoring | 20 |
| PEG 400 | Solvent | 11.9 |
| PEG 4000 | Binder | 5.0 |
| polyethylene glycol 12-hydroxystearate | Surfactant | 5.0 |
| Caprylic/capric triglyceride | Solvent/lubricant | 1.0 |
| Glycerin | Humectant | 3.0 |
| Potassium Sorbate | Preservative | 0.3 |

TABLE 30

| Ingredients | Function | % (w/w) |
|---|---|---|
| Antimicrobial IgY Antibody | Active | 13.6 |
| Soy protein fines | Filler | 19.2 (QS) |
| Corn Starch | Filler | 20 |
| beef flavor | Flavoring | 20 |
| PEG 400 | Solvent | 11.9 |
| PEG 4000 | Binder | 5.0 |
| polyethylene glycol 12-hydroxystearate | Surfactant | 5.0 |
| Caprylic/capric triglyceride | Solvent/lubricant | 1.0 |
| Glycerin | Humectant | 4.0 |
| Potassium Sorbate | Preservative | 0.3 |

TABLE 31

| Ingredients | Function | % (w/w) |
|---|---|---|
| Antimicrobial IgY Antibody | Active | 13.6 |
| Soy protein fines | Filler | 24.2 (QS) |
| Corn Starch | Filler | 15 |
| beef flavor | Flavoring | 20 |
| PEG 400 | Solvent | 11.9 |
| PEG 4000 | Binder | 5.0 |
| polyethylene glycol 12-hydroxystearate | Surfactant | 5.0 |
| Caprylic/capric triglyceride | Solvent/lubricant | 1.0 |
| Glycerin | Humectant | 4.0 |
| Potassium Sorbate | Preservative | 0.3 |

TABLE 32

| Ingredients | Function | % (w/w) |
|---|---|---|
| Antimicrobial IgY antibody | Active | 1.875 |
| Milbemycin oxime | Active | 0.375 |
| Corn starch | Filler | 20.0 |
| Soy protein fines | Filler | 28.3 (QS) |
| beef flavor | Flavoring | 25.0 |
| Povidone K-30 | Binder | 6.0 |
| PEG 400 | Solvent | 12.0 |
| polyethylene glycol 12-hydroxystearate | Surfactant | 3.0 |
| Caprylic/capric triglyceride | Solvent/lubricant | 3.0 |
| Potassium sorbate | Preservative | 0.3 |
| BHT | Antioxidant | 0.14 |

TABLE 33

| Ingredients | Function | % (w/w) |
|---|---|---|
| Moxidectin | Active | 0.03 |
| Milbemycin oxime | Active | 0.375 |
| Antimicrobial IgY antibody | Active | 1.875 |
| Soy protein fines | Filler | 23 (QS) |
| Starch | Filler | 21 |
| beef flavor | Flavoring | 20 |
| Povidone K-30 | Binder | 2.75 |
| PEG 400 | Solvent | 7.1 |
| polyethylene glycol 12-hydroxystearate | Surfactant | 3.1 |
| PEG 4000 | Binder | 6.35 |
| Caprylic/capric triglyceride | Solvent/lubricant | 3.15 |
| Glycerin | Humectant | 10.0 |
| Potassium sorbate | Preservative | 0.3 |
| BHT | Antioxidant | 0.14 |
| Citric acid | Preservative | 0.5 |

### Example 2

### Evaluation of the Effectiveness of IgY Antibodies on the Reduction of Plaque, Calculus, Gingivitis and Halitosis in Adult Dogs

The objective of this study is to evaluate the effectiveness of IgY antibodies on the reduction of dental plaque, calculus, gingivitis and halitosis in adult Beagle dogs.

Beagles are either given 25 mg (per application) of IgYa antibodies (Group 1), IgYb antibodies (Group 2), or gel formulation vehicle alone (Group 3; control).

Pre-Test Phase (Day −7 to Day 0): A pre-test phase of seven days is conducted before the initiation of the 31-day treatment period. During the pre-test phase, the dogs selected for this study are weighed and fed the control diet, Purina Dog Chow a kibble, fed dry only. Each animal will have its teeth scaled and polished upon initiation of the pre-test phase (Day −7). On Day 0, halitosis, plaque and gingivitis are evaluated. The scores obtained are indicative of the rate of plaque buildup for each animal when being fed the control diet only. Animals are stratified into 4 groups based on plaque scores. This procedure is performed in an attempt to reduce the variability of scores during the test/treatment phase. Following the dental scoring on Day 0, each animal will undergo a dental cleaning and polishing procedure. After this procedure a disclosing agent of 2% Eosin is applied to all test teeth and an oral examination is performed to ensure there is no remaining plaque or calculus buildup and a clean mouth is used for the start of the study.

Test Phase (Day 0 to Day 31): On Days 0-31 all dogs are fed control diet only. Dogs assigned to Group 1 are treated with test article IgYa based gel, dogs assigned to Group 2 are treated with test article IgYb based gel and dogs assigned to Group 3 are treated with the control article plain gel every other day, beginning on Day 1. The gel is applied along the gingival margin on the upper and lower jaw. Dogs assigned to Group 4 are fed a control diet and will not be treated. On Day 31, each animal will undergo halitosis, gingivitis and calculus evaluations by one technician, and a plaque evaluation by a second technician.

The dates for the initiation of the pre-test phase and the test phase are staggered due to the time required for teeth scaling and polishing. Start dates are consistent for the number of animals between the groups, and each animal will have a 7-day pre-test phase followed by a 31-day test phase.

TABLE 34
Study activities

| Day | Activity |
|---|---|
| −8 | Body weights |
| −7 | Cleaning/polishing teeth |
| −1 | Body weights |
| 0 | Dental Evaluation followed by cleaning/polishing |
| 1 | Oral treatment (Groups 1, 2 and 3) |
| 3 | Oral treatment (Groups 1, 2 and 3) |
| 5 | Oral treatment (Groups 1, 2 and 3) |
| 6 | Body weights |
| 7 | Oral treatment (Groups 1, 2 and 3) |
| 9 | Oral treatment (Groups 1, 2 and 3) |
| 11 | Oral treatment (Groups 1, 2 and 3) |
| 13 | Oral treatment (Groups 1, 2 and 3) |
| | Body weights |
| 15 | Oral treatment (Groups 1, 2 and 3) |
| 17 | Oral treatment (Groups 1, 2 and 3) |
| 19 | Oral treatment (Groups 1, 2 and 3) |
| 20 | Body weights |
| 21 | Oral treatment (Groups 1, 2 and 3) |
| 23 | Oral treatment (Groups 1, 2 and 3) |
| 25 | Oral treatment (Groups 1, 2 and 3) |
| 27 | Oral treatment (Groups 1, 2 and 3) |
| | Body weights |
| 29 | Oral treatment (Groups 1, 2 and 3) |
| 31 | Dental Evaluation |

Description Purpose-bred Beagle dogs. Number. A minimum of 40 animals one year of age or older are used. Dogs are split into four groups of ten. The groups will generally be determined by stratifying the animals according to their plaque scores from Day 0 and assigning animals with similar plaque scores to each of the study groups. This method is used in an attempt to reduce the variability among the study groups. An equal number of dogs is assigned to each of the study groups.

Criteria for Animals Used. Healthy adult Beagle dogs meeting the above description are eligible for the study. Before study initiation, the dogs will undergo an oral cavity examination for normal occlusion and the presence of all test teeth. The initial oral examination will include a visual check for the presence of a significant furcation defect (loss of bone between the roots of multi-rooted teeth). Dogs with any complete furcation defect (i.e. a periodontal probe can be passed from the buccal surface and seen to emerge on the palatal/lingual surface) in any of required test teeth will not be included in this study.

Acclimation of Test Animals. Dogs are housed as intended for this study prior to study initiation.

Methods. Weights. Individual body weights are measured and recorded weekly throughout the study (Days −8, −1, 6, 13, 20 and 27).

Control Diet Fed During the Study (Day −7 to Day 31). Throughout the study, Purina Dog Chow, a kibble, fed dry, are supplied to each animal once daily for a minimum of 1 hour. Dogs are fed according to ideal body condition. Food consumption is recorded.

Application of the Test and control article. Dogs assigned to group 1, 2 and 3 are administered the appropriate treatment every other day throughout the course of the study (15 total treatments). The test article is applied approximately 2 hours after the control diet has been removed. Test articles and control article are applied along the top and bottom gingival line on both sides of the mouth.

TABLE 35
Study design

| | DOGS | | | |
|---|---|---|---|---|
| | PER | | | |
| GROUP | GROUP | TEST/GROUP | ROUTE/DOSE | FREQUENCY |
| 1 | 10 | IgYa based gel | Oral application | Every other |
| | | | 25 mg of IgY | day for 30 days |
| 2 | 10 | IgYb based gel | Oral application | (total 15 |
| | | | 25 mg of IgY | treatments) |
| 3 | 10 | Plain gel* | Oral application | |
| | | | plain gel | |
| 4 | 10 | — | — | — |

** A preparation of CARBIGEN M (CARBOPOL 947) at an optimal viscosity will be used as the vehicle (gel) for mixture with the IgY. All treatments will be prepared, dog side, prior to administration.
IgYa - purified anti-chimeric gingipain IgY Approximately 4.6 g of antibody were produced from 56 eggs. Frozen suspension of at a concentration of 370 ml at 12.6 mg/ml.
IgYb - purified Muno-Igy - no immunization (i.e. native IgY produced by the hens)

Dental Procedures. Pre-Test Phase. Each animal has its teeth scaled and polished on Day −7. On Day 0 each animal has halitosis, plaque and gingivitis evaluated which is followed by another dental cleaning and polishing. Halitosis, plaque and gingivitis assessments and dental cleanings/polishing are performed using general anesthesia. After the cleaning procedure a disclosing agent of 2% Eosin is applied to all test teeth and an oral examination is performed to ensure there is no remaining plaque or calculus buildup and a clean mouth is used for the start of the study. Test Phase. On Day 31, evaluation of gingivitis, calculus, plaque and halitosis are performed on each animal. Dental evaluations are performed under general anesthesia. Halitosis and gingivitis are evaluated first, and then calculus accumulation is scored prior to applying the disclosing agent for plaque evaluation. The examination of teeth will include the buccal surfaces and both sides of the mouth. Review is limited to these nine teeth: upper jaw—incisor 3 (13), canine (C), premolar 3 (P3), premolar 4 (P4) and molar 1 (M1); lower jaw—canine (C), premolar 3 (P3), premolar 4 (P4), molar 1 (M1).² Qualified dental scorers will not be involved in any study-related activities apart from dental scoring. Additionally, animals are selected in random order on Day 31, so that the scorers are unaware of each animal's treatment when scoring the teeth. Dental Observations. The presence of inflammation, ulceration or laceration anywhere in the oral cavity is recorded for each dog during dental scoring on Days 0 and 30.

Clinical Observations/Measurements.

Physical Examinations. Each dog is evaluated as to general health including oral health, body and hair coat condition and comments are recorded. Two milliliters of blood is taken from each dog to test for BUN, PCV, and TP prior to treatment initiation if these data have not been updated within the last 6 months.

Observations. Qualified personnel will perform clinical observations twice daily in accordance with standard procedures.

Dental Parameter Evaluation.

Halitosis. Oral malodor is evaluated using a halimeter. Readings are obtained by putting the straw end of the halimeter into the dog's mouth between the cheek and jaw. A pocket is made and the lips are closed around the straw to assure accurate readings. One reading is taken on each side of the mouth, and is recorded in ppb.

Gingivitis. Gingivitis, defined as the inflammation of the gums surrounding the teeth, is evaluated by the modified gingival index based on Lobene et al. (1986), which is herein incorporated by reference in its entirety. The MGI scoring system² is as set out in Table 36.

TABLE 36
MGI scoring system details
Gingivitis Scoring Method

| Score | Degree of Inflammation |
|---|---|
| 0 | Absence of inflammation |
| 1 | Mild inflammation; slight change in color, little change in texture |
| | of any portion of the marginal or papillary gingival unit |
| 2 | Mild inflammation; criteria as above but involving the entire |
| | marginal or papillary gingival unit |
| 3 | Moderate inflammation; glazing, redness, edema, and/or |
| | hypertrophy of the marginal or papillary gingival unit |
| 4 | Severe inflammation; marked redness, edema and/or hypertrophy |
| | of the marginal or papillary gingival unit, spontaneous |
| | bleeding, congestion, or ulceration |

Each tooth is assigned a numerical score based on the degree of inflammation. The sum of the teeth scores are divided by the number of teeth examined (18) to obtain a whole mouth mean gingivitis score for each animal.

Calculus. Calculus is defined as the calcium salts secreted in saliva that are deposited on the surface of the teeth as a hard substance that is resistant to removal by chewing or brushing. Calculus (tartar) is to be recorded after air-drying the tooth surface. Calculus is scored quantitatively using modifications of a method developed by Schiff. Each tooth is assigned a numerical score based on the percentage of calculus coverage (see calculus scoring method below). The sum of the teeth scores are divided by the number of teeth examined (18) to obtain a whole mouth mean calculus score for each animal.¹

TABLE 37
Calculus Scoring Method
Calculus Scoring Method

| Score | % of Calculus Coverage |
|---|---|
| 0 | No calculus detected |
| 1 | 1-24 |
| 2 | 25-49 |
| 3 | 50-74 |
| 4 | 75-100 |

Plaque. Plaque is defined as the soft, bacteria-rich layer that rapidly forms on the surface of the teeth. Plaque is evaluated using a modification of the Quigley and Hein (1962) (Turesky, 1970) plaque index. The extent of plaque and plaque thickness is determined by placing a disclosing agent on the teeth and rinsing the excess off with water. The whole tooth is scored: The teeth are visually halved by visualizing midway between the gingival margin and the tooth cusp. Each half is given a score for the percentage of coronal surface covered with plaque and thickness of plaque. The score for each half is calculated by multiplying the coverage and thickness scores. Gingival and occlusal scores are then added together for a whole tooth score. The sum of the tooth scores are divided by the number of teeth examined (18) to obtain a whole mouth mean plaque score for each animal.¹

TABLE 38
Plaque Scoring Method
Plaque Scoring Method

| Score | % of Plaque Coverage |
|---|---|
| 0 | No plaque detected |
| 1 | 1-24 |
| 2 | 25-49 |
| 3 | 50-74 |
| 4 | 75-100 |

| Score | Plaque Thickness | Disclosing Agent Color |
|---|---|---|
| 1 | Light | Pink to Light Red |
| 2 | Medium | Red |
| 3 | Heavy | Dark Red |

Variable(s) to be measured. Primary Parameter 1: The effects of the IgY antibodies on canine dental plaque, calculus, gingivitis and halitosis. Secondary Parameter 1. Food consumption (control diet); Secondary Parameter 2: Body weight changes; Secondary Parameter 3: Adverse reactions, daily observations.

Statistical Methodology. Individual t-tests, mean scores, standard error and standard deviation functions are performed on all dental parameters. Results obtained for treated groups are compared with the results obtained for Group 4 (untreated group). Means, standard errors and standard deviations will also be obtained for food consumption and body weights/weight changes.

### STUDY REFERENCES

¹Logan E I, Boyce E N. Oral Health Assessment in Dogs: Parameters and Methods. J. Vet. Dent. 1994 August; 11(2): 58-63.
²The Veterinary Oral Health Council, A System for Recognition of Effectiveness of Veterinary Dental Products: Protocol and Submissions, Haddonfield, N J, 2014.
³Holmstrom, S. E., Frost, P. and Eisner, E. R. Veterinary Dental Techniques for the Small Animal Practitioner, 2^{nd} ed. W. B. Saunders, Philadelphia, Pa. 1998: p. 482.

Conclusion. Dogs treated with anti-chimeric gingipain IgY showed significant improvement across all oral health parameters, when compared to dogs treated with Muno-IgY (i.e. endogenous/native IgY from the yolks of un-immunized hens) or gel-only.

The invention will now be described in the following numbered paragraphs:

1. A composition for reducing or eliminating oral health compromising (OHC) microorganisms, comprising:
a. a safe and effective amount of an antimicrobial IgY antibody, produced in the egg of an avian animal;
b. a veterinarily or pharmaceutically acceptable vehicle or carrier; and
c. optionally comprising all the naturally-occurring components of said egg; and

wherein the IgY antibody was produced using, or raised against and reacts to, an immunogenic portion of any one or more of the polypeptide sequences as set forth in SEQ ID NOs: 110-113, 120, 123-125, 130-133, 135-138, 143-148, 221-224 or 227-349; or

wherein the IgY antibody was produced using, or raised against, a polypeptide comprising, consisting of or consisting essentially of one or more of the following: SEQ ID NO:221; residues 1-184 of SEQ ID NO:221; residues 1-290 of SEQ ID NO:221; residues 65-184 of SEQ ID NO:221; residues 65-290 of SEQ ID NO:221; residues 65-419 of SEQ ID NO:221; residues 192-290 of SEQ ID NO:221; residues 192-419 of SEQ ID NO:221; residues 147-419 of SEQ ID NO:221; SEQ ID NO:225; or SEQ ID NO:226; or

wherein the IgY antibody was produced using, or raised against, a polypeptide comprising, consisting of or consisting essentially of one or more of the following: residues 65-184 of SEQ ID NO:221; residues 65-290 of SEQ ID NO:221; or residues 192-290 of SEQ ID NO:221; or

wherein the IgY antibody was produced using, or raised against, a polypeptide comprising, consisting of or consisting essentially of one or more of the following: residues 65-184 of SEQ ID NO:221, residues 65-290 of SEQ ID NO:221 and residues 192-290 of SEQ ID NO:221; or

wherein the IgY antibody was produced using, or raised against, a polypeptide comprising, consisting of or consisting essentially of SEQ ID NO:222;

wherein the IgY antibody was produced using, or raised against, a polypeptide comprising, consisting of or consisting essentially of a sequence having greater than 60, 70, 80 or 90% identity to the sequence as set forth in SEQ ID NO:221, 222, 223 or 224.

2. The composition of paragraph 1, formulated as a soft chewable veterinary composition, wherein the carrier comprises one or more fillers, at least one flavoring agent, at least one binder, one or more solvents, one or more surfactants, at least one humectant, optionally an antioxidant, and optionally a preservative.
3. The composition of paragraph 2, wherein the one or more fillers is soy protein fines, corn starch, or a mixture thereof
4. The composition of paragraph 3, wherein the binder is polyvinylpyrrolidone or a polyethylene glycol, or a combination thereof.
5. The composition of paragraph 4, wherein the solvent is a liquid polyethylene glycol or a caprylic/capric triglyceride, or a combination thereof
6. The composition of paragraph 4, wherein the surfactant is polyethylene glycol hydroxystearate.
7. The composition of paragraph 4, wherein the humectant is glycerin.
8. The composition of paragraph 4, wherein the flavoring agent is an artificial meat or beef flavor.
9. The composition of paragraph 4, comprising:
a. a filler selected from corn starch, pre-gelatinized corn starch, corn gluten meal and soy protein fines, or a combination thereof
b. a solvent selected from liquid polyethylene glycols, propylene glycol, propylene carbonate, caprylic/capric triglycerides, caprylic/capric/linoleic triglycerides, caprylic/capric/succinic triglycerides, propylene glycol dicaprylate/dicaprate, glycerol caprylate/caprate and polyglycolized glycerides, or a combination thereof;
c. a binder selected from polyvinylpyrrolidone, polyethylene glycols, co-polymers of vinyl acetate and vinylpyrrolidone, potato starch and corn starch, or a combination thereof
d. a humectant selected from glycerol, propylene glycol, cetyl alcohol, glycerin monostearate and polyethylene glycols, or a combination thereof
e. a surfactant selected from glyceryl monooleate, polyoxyethylene sorbitan fatty acid esters, sorbitan esters, polyvinyl alcohol, polysorbates, sodium lauryl sulfate, co-polymers of ethylene oxide and propylene oxide, propylene glycol monolaurate, glycerol caprylate/caprate, polyglycolized glycerides and polyethylene glycol hydroxystearate, or a combination thereof and
f. a natural or artificial beef or meat flavor.
   10. The composition of paragraph 9, wherein the composition comprises IgY antibody at a concentration of about 1% to about 20% by weight.
   11. The composition of paragraph 10, wherein:
a. the filler is a combination of corn starch and soy protein fines and is present at a concentration of about 30% to about 50% (w/w);
b. the solvent is a mixture of liquid polyethylene glycol and caprylic/capric triglycerides and is present at a concentration of about 5% to about 20% (w/w);
c. the binder is polyethylene glycol or polyvinylpyrrolidone, or a combination thereof, and is present at a concentration of about 5% to about 15% (w/w);
d. the humectant is glycerin and is present at a concentration of about 5% to about 20%;
e. the surfactant is polyethylene glycol 12-hydroxystearate or polyoxyl hydrogenated castor oil and is present at a concentration of about 1% to about 5% (w/w).
   12. The composition of paragraph 12, wherein the IgY antibody is present at a concentration of about 1% to about 5% by weight.
   13. The composition of paragraph 12, wherein the IgY antibody is present at a concentration of about 10% to about 20% by weight.
   14. The composition of paragraph 1, wherein the composition comprises a combination of at least one antimicrobial IgY antibody, active against oral health compromising (OHC) microorganisms, and at least one systemically-acting active agent selected from one or more arthropodicidal isoxazolines, one or more macrocyclic lactones, one or more spinosyn compounds, one or more spinosoid compounds, one or more benzimidazoles, levamisole, pyrantel, morantel, praziquantel, closantel, clorsulon, one or more amino acetonitrile active agents, one or more insect growth regulators, one or more neonicotinoids and one or more aryloazol-2-yl cyanoethylamino active agents, or a combination of thereof
   15. The composition of paragraph 14, wherein the macrocyclic lactone is eprinomectin, ivermectin, selamectin, milbemectin, milbemycin D, milbemycin oxime, or moxidectin, or a combination thereof
   16. A method for the treatment and/or prevention of an oral health compromising (OHC) microorganism infection in an animal's oral cavity comprising administering to the animal an effective amount of the composition of paragraph 1.
   17. The method of paragraph 20, wherein the OHC microorganism is S. mutans, S. salivarius, S. mutans, S. sanguinis and S. sobrinus, P. gulae, P. salivosa, P. gingivalis and P. denticanis.
   18. Use of an antimicrobial IgY antibody of paragraph 1 in the manufacture of a soft chewable veterinary composition for the treatment and/or prevention of an OHC microorganism infection in an animal.
   19. A method of reducing the occurrence of dental caries or other oral health diseases or pathologies in an animal comprising the steps of:
a. selecting an animal with or at risk of developing oral health diseases or pathologies, including dental caries; and
b. contacting the oral cavity, and particularly the teeth, of the animal with a therapeutically effective amount of the antimicrobial IgY antibody-containing composition of paragraph 1; and
   wherein the antibody is present in a safe and effective amount to reduce or eliminate adherence of one or more oral health compromising (OHC) microorganism(s) that cause oral or dental diseases or pathologies, thereby reducing the occurrence of dental caries or other oral health diseases or pathologies.
      20. The method of paragraph 19 wherein the IgY antibody is specific for at least one of the following OHC microorganisms: S. mutans, S. salivarius, S. mutans, S. sanguinis and S. sobrinus, P. gulae, P. salivosa, P. gingivalis and P. denticanis.
      21. The method of paragraph 19, wherein the therapeutically effective amount of the antimicrobial IgY antibody reduces the number or adherence of the OHC S. mutans, S. salivarius, S. mutans, S. sanguinis and S. sobrinus, P. gulae, P. salivosa, P. gingivalis and P. denticanis.
      22. The method of paragraph 19, wherein the OHC is S. mutans.
      23. The method of paragraph 19, wherein the therapeutically effective amount of the IgY antibody disrupts dental plaque in the oral cavity of the animal.
      24. The method of paragraph 19, wherein the IgY antibody is an oral treatment form selected from a toothpaste, mouth rinse, gel, foam, varnish, polish, floss, dental tray, dental strip, copolymer membrane, and slow release bead.
      25. The method of paragraph 19, wherein the IgY antibody is present in the oral supplement in a dosage of less than 1 mg/ml.
      26. The method of paragraph 19, wherein the IgY antibody is present in the oral supplement in a dosage of less than 100 μg/ml.
      27. The method of paragraph 19, wherein the IgY antibody is present in the oral supplement in a dosage of 50 μg/ml.
      28. The method of paragraph 19, wherein the IgY antibody is present in the oral supplement in a dosage of 25 μg/ml.
      29. An oral treatment form comprising the IgY-containing composition of paragraph 1.
      30. The oral treatment form of paragraph 29, wherein the oral treatment form is selected from a toothpaste, mouth rinse, gel, foam, varnish, polish, floss, dental strip, copolymer membrane, and slow release bead.
      31. The oral treatment form of paragraph 30, wherein the IgY antibody is present in an amount of less than 1 mg.
      32. The oral treatment form of paragraph 30, wherein the IgY antibody is present in an amount of less than 100 μg.
      33. The oral treatment form of paragraphs 30, wherein the IgY antibody is present in an amount of 50 μg.
      34. The oral treatment form of paragraphs 30, wherein the IgY antibody is present in an amount of 25 μg.
      35. A kit comprising the oral treatment form of paragraph 30 and an applicator.
      36. The kit of paragraph 35, wherein the applicator is a toothbrush or dental tray.
      37. A composition, comprising:
a. a transfer agent;
b. a barrier material;
c. an antimicrobial IgY antibody, which is active in reducing or eliminating the numbers of one or more oral health compromising (OHC) microorganism from the oral cavity, including the teeth, of an animal.
   38. The composition of paragraph 37, wherein:
a. said transfer agent is present in an amount of 0.25 to 25 wt. %, based on the total weight of said transfer agent and said barrier material; and
b. said barrier material is present in an amount of 75 to 99.75 wt. %, based on the total weight of said transfer agent and said barrier material.
   39. The composition of paragraph 38, wherein said transfer agent is selected from lecithin, cetyl amine, N-tallow-1,3-propanediamine, hexetidine, and cetylpyridinium halide.
   40. The composition of paragraph 38, wherein:
a. said transfer agent is present in an amount of 3 to 5 wt. %, based on the total weight of said transfer agent and said barrier material;
b. said barrier material is present in an amount of 95 to 97 wt. %, based on the total weight of said transfer agent and said barrier material.
   41. The composition of paragraph 38, wherein said transfer agent is hexetidine.
   42. The composition of paragraph 38, wherein said barrier material is selected from natural waxes, synthetic waxes, silicone-based polymers, and fluoropolymers.
   43. The composition of paragraph 41, wherein said barrier material is a microcrystalline wax.
   44. The composition of paragraph 42, which is a film, a toothpaste, or a chewing gum.
   45. The composition of paragraph 40, wherein said transfer agent is cetylpyridinium halide.
   46. The composition of paragraph 45, wherein said barrier material is selected from natural waxes, synthetic waxes, silicone-based polymers, and fluoropolymers.
   47. The composition of paragraph 45 wherein said barrier material is a microcrystalline wax.
   48. The composition of paragraph 45, which is a toothpaste or a chewing gum.
   49. A composition, comprising:
a. a transfer agent;
b. a barrier material; and
c. an antimicrobial IgY antibody active agent, which is active in reducing or eliminating the numbers of one or more oral health compromising (OHC) microorganism from the oral cavity, including the teeth, of an animal;
wherein: said transfer agent is present in an amount of 0.25 to 25 wt. %, based on the total weight of said transfer agent, said barrier material, and said active agent;
said barrier material is present in an amount of 50 to 99.50 wt. %, based on the total weight of transfer agent, said barrier material, and said active agent;
said active agent is present in an amount of 0.25 to 25 wt. %, based on the total weight of transfer agent, said barrier material, and said active agent and said transfer agent is selected from cetyl amine, N-tallow-1,3-propanediamine, hexetidine, and cetylpyridinium halide.
   50. The composition of paragraph 49, wherein:
a. said transfer agent is present in an amount of 1 to 5 wt. %, based on the total weight of said transfer agent, said barrier material, and said active agent;
b. said barrier material is present in an amount of 85 to 98 wt. %, based on the total weight of said transfer agent, said barrier material, and said active agent; and
c. said active agent is present in an amount of 1 to 10 wt. %, based on the total weight of said transfer agent, said barrier material, and said active agent.
   51. The composition of paragraph 50, wherein said transfer agent is hexetidine.
   52. The composition of paragraph 51, wherein said barrier material is selected from natural waxes, synthetic waxes, silicone-based polymers, and fluoropolymers.
   53. The composition of paragraph 52, wherein said barrier material is a microcrystalline wax.
   54. The composition of paragraph 52, which is film, a toothpaste, or a chewing gum.
   55. The composition of paragraph 50, wherein said transfer agent is cetylpyridimum halide.
   56. The composition of paragraph 55, wherein said barrier material is selected from natural waxes, synthetic waxes, silicone-based polymers, and fluoropolymers.
   57. The composition of paragraph 56, wherein said barrier material is a microcrystalline wax.
   58. The composition of paragraph 57, which is a film, a toothpaste, or a chewing gum.
   59. A method of protecting teeth, comprising treating teeth with a composition, wherein said composition comprises:
a. a transfer agent; and
b. a barrier material;
c. an antimicrobial IgY antibody active agent, which is active in reducing or eliminating the numbers of one or more oral health compromising (OHC) microorganism from the oral cavity, including the teeth, of an animal;
wherein:
said transfer agent is present in an amount of 0.25 to 25 wt. %, based on the total weight of said transfer agent and said barrier material;
said barrier material is present in an amount of 75 to 99.75 wt. %, based on the total weight of said transfer agent and said barrier material; and
said transfer agent is selected from cetyl amine, N-tallow-1,3-propanediamine, hexetidine, and cetylpyridinium halide.
   60. The method of paragraph 59, wherein:
said transfer agent is present in an amount of 3 to 5 wt. %, based on the total weight of said transfer agent and said barrier material;
said barrier material is present in an amount of 95 to 97 wt. %, based on the total weight of said transfer agent and said barrier material.
   61. The method of paragraph 60, wherein said transfer agent is hexetidine.
   62. The method of paragraph 61, wherein said barrier material is selected from natural waxes, synthetic waxes, silicone-based polymers, and fluoropolymers.
   63. The method of paragraph 62, wherein said barrier material is a microcrystalline wax.
   64. The method of paragraph 62, wherein said composition is in the form of a film, a toothpaste, or a chewing gum.
   65. The method of paragraph 60, wherein said transfer agent is cetylpyridinium halide.
   66. The method of paragraph 65, wherein said barrier material is selected from natural waxes, synthetic waxes, silicone-based polymers, and fluoropolymers.
   67. The method of paragraph 65, wherein said barrier material is a microcrystalline wax.
   68. The method of paragraph 67, wherein said composition is in the form of a film, a toothpaste, or a chewing gum.
   69. A dental delivery system, comprising a substrate coated with a composition, wherein said composition comprises:
a. a transfer agent;
b. optionally a barrier material; and
c. an antimicrobial IgY antibody active agent, which is active in reducing or eliminating the numbers of one or more oral health compromising (OHC) microorganism from the oral cavity, including the teeth, of an animal;
wherein:
said transfer agent is present in an amount of 0.25 to 25 wt. %, based on the total weight of said transfer agent and said barrier material;
said barrier material, when present, is present in an amount of 75 to 99.75 wt. %, based on the total weight of said transfer agent and said barrier material; and
said transfer agent is selected from lecithin, cetyl amine, N-tallow-1,3-propanediamine, hexetidine, and-cetylpyridinium halide.
   70. The dental delivery system of paragraph 69, wherein:
a. said transfer agent is present in an amount of 3 to 5 wt. %, based on the total weight of said transfer agent and said barrier material;
b. said barrier material is present in an amount of 95 to 97 wt. %, based on the total weight of said transfer agent and said barrier material.
   71. The dental delivery system of paragraph 69, wherein said transfer agent is hexetidine.
   72. The dental delivery system of paragraph 69, wherein said barrier material is selected from natural waxes, synthetic waxes, silicone-based polymers, and fluoropolymers.
   73. The dental delivery system of paragraph 72, wherein said barrier material is a microcrystalline wax.
   74. The dental delivery system of paragraph 73, wherein said composition is in the form of a film, a toothpaste, or chewing gum.
   75. The dental delivery system of paragraph 74, wherein said transfer agent is cetylpyridinium halide.
   76. The dental delivery system of paragraph 75, wherein said barrier material is selected from natural waxes, synthetic waxes, silicone-based polymers, and fluoropolymers.
   77. The dental delivery system of paragraph 75, wherein said barrier material is a microcrystalline wax.
   78. The dental delivery system of paragraph 75, wherein said composition is in the form of a film, a toothpaste, or chewing gum.
   79. A dental delivery system, comprising a substrate coated with a composition, wherein said composition comprises:
a. a transfer agent;
b. a barrier material; and
c. an antimicrobial IgY antibody active agent, which is active in reducing or eliminating the numbers of one or more oral health compromising (OHC) microorganism from the oral cavity, including the teeth, of an animal;
wherein:
said transfer agent is present in an amount of 0.25 to 25 wt. %, based on the total weight of said transfer agent, said barrier material, and said active agent;
said barrier material is present in an amount of 50 to 99.50 wt. %, based on the total weight of transfer agent, said barrier material, and said active agent;
said active agent is present in an amount of 0.25 to 25 wt. %, based on the total weight of transfer agent, said barrier material, and said active agent and said transfer agent is selected from cetyl amine, N-tallow-1,3-propanediamine, hexetidine, and cetylpyridinium halide.
   80. The dental delivery system of paragraph 79, wherein:
a. said transfer agent is present in an amount of 1 to 5 wt. %, based on the total weight of said transfer agent, said barrier material, and said active agent;
b. said barrier material is present in an amount of 85 to 98 wt. %, based on the total weight of said transfer agent, said barrier material, and said active agent; and
c. said active agent is present in an amount of 1 to 10 wt. %, based on the total weight of said transfer agent, said barrier material, and said active agent.
   81. The dental deliver system of paragraph 80, wherein said transfer agent is hexetidine.
   82. The dental delivery system of paragraph 81, wherein said barrier material is selected from natural waxes, synthetic waxes, silicone-based polymers, and fluoropolymers.
   83. The dental delivery system of paragraph 81, wherein said barrier material is a microcrystalline wax.
   84. The dental delivery system of paragraph 81, wherein said active agent is chlorhexidine.
   85. The dental delivery system of paragraph 81, wherein said composition is in the form of a film, a toothpaste, or chewing gum.
   86. The dental delivery system of paragraph 80, wherein said transfer agent is cetylpyridinium halide or lecithin.
   87. The dental delivery system of paragraph 86, wherein said barrier material is selected from natural waxes, synthetic waxes, silicone-based polymers, and fluoropolymers.
   88. The dental delivery system of paragraph 86, wherein said barrier material is a microcrystalline wax.
   89. The dental delivery system of paragraph 86, wherein said active agent is selected from hexetidine and chlorhexidine.
   90. The dental delivery system of paragraph 86, wherein said composition is in the form of a film, a toothpaste, or chewing gum.
   91. A method for producing antimicrobial IgY antibodies, which are effective in reducing or eliminating oral health compromising (OHC) microorganism, comprising the step of administering to a hen at least one OHC microorganism-associated polypeptide having a sequence having at least 90% identity to an immunologically sufficient portion of a sequence as set forth in any one of the following sequences: SEQ ID NO: 227-349.
   92. A composition comprising one or more antimicrobial IgY of paragraph 91.
   93. A method of treatment comprising the step of administering one or more antimicrobial IgY of paragraph 91.
   94. A method for producing antimicrobial IgY antibodies, which are effective in reducing or eliminating oral health compromising (OHC) microorganism, comprising the step of administering to a hen at least one OHC microorganism-associated polypeptide having a sequence having at least 90% identity to an immunologically sufficient portion of a sequence as set forth in any one of the following SEQ ID NOs: 110-113, 120, 123-125, 130-133, 135-138, 143-148, 221-224 or 227-349.
   95. A composition comprising one or more antimicrobial IgY of paragraph 94.
   96. A method of treating an animal in need thereof comprising the step of administering one or more antimicrobial IgY of paragraph 94.
   97. A method for alleviating sensitivity of teeth, comprising sensitive teeth with a composition, wherein said composition comprises:
a. a transfer agent; and
b. a barrier material;

wherein:

said transfer agent is present in an amount of 0.25 to 25 wt. %, based on the total weight of said transfer agent and said barrier material;

said barrier material is present in an amount of 75 to 99.75 wt. %, based on the total weight of said transfer agent and said barrier material; and

said transfer agent is selected from lecithin, cetyl amine, N-tallow-1,3-propanediamine, hexetidine, and cetylpyridinium halide.

What is claimed is:

1. A composition for reducing or eliminating oral health compromising (OHC) microorganisms, comprising:
a. a safe and effective amount of an antimicrobial IgY antibody, produced in the egg of an avian animal;
b. a veterinarily or pharmaceutically acceptable vehicle or carrier; and
c. optionally comprising all the naturally-occurring components of said egg; and
wherein the IgY antibody was produced using, or raised against and reacts to, an immunogenic portion of any one or more of the polypeptide sequences as set forth in SEQ ID NOs: 110-113, 120, 123-125, 130-133, 135-138, 143-148, 221-224 or 227-349; or
wherein the IgY antibody was produced using, or raised against, a polypeptide comprising, consisting of or consisting essentially of one or more of the following: SEQ ID NO:221; residues 1-184 of SEQ ID NO:221; residues 1-290 of SEQ ID NO:221; residues 65-184 of SEQ ID NO:221; residues 65-290 of SEQ ID NO:221; residues 65-419 of SEQ ID NO:221; residues 192-290 of SEQ ID NO:221; residues 192-419 of SEQ ID NO:221; residues 147-419 of SEQ ID NO:221; SEQ ID NO:225; or SEQ ID NO:226; or
wherein the IgY antibody was produced using, or raised against, a polypeptide comprising, consisting of or consisting essentially of one or more of the following: residues 65-184 of SEQ ID NO:221; residues 65-290 of SEQ ID NO:221; or residues 192-290 of SEQ ID NO:221; or
wherein the IgY antibody was produced using, or raised against, a polypeptide comprising, consisting of or consisting essentially of one or more of the following: residues 65-184 of SEQ ID NO:221, residues 65-290 of SEQ ID NO:221 and residues 192-290 of SEQ ID NO:221; or
wherein the IgY antibody was produced using, or raised against, a polypeptide comprising, consisting of or consisting essentially of SEQ ID NO:222;
wherein the IgY antibody was produced using, or raised against, a polypeptide comprising, consisting of or consisting essentially of a sequence having greater than 60, 70, 80 or 90% identity to the sequence as set forth in SEQ ID NO:221, 222, 223 or 224.

2. The composition of claim 1, wherein the IgY antibody was produced using, or raised against, a polypeptide comprising, consisting of or consisting essentially of a sequence having greater than 60% identity to the sequence as set forth in SEQ ID NO:221, 222, 223 or 224.

3. The composition of claim 1, wherein the composition is formulated as a gel.

4. The composition of claim 1, wherein the composition is formulated as a soft chewable veterinary composition, wherein the carrier comprises one or more fillers, at least one flavoring agent, at least one binder, one or more solvents, one or more surfactants, at least one humectant, optionally an antioxidant, and optionally a preservative;
wherein the one or more fillers is soy protein fines, corn starch, or a mixture thereof;
wherein the binder is polyvinylpyrrolidone or a polyethylene glycol, or a combination thereof;
wherein the solvent is a liquid polyethylene glycol or a caprylic/capric triglyceride, or a combination thereof;
wherein the surfactant is polyethylene glycol hydroxystearate;
wherein the humectant is glycerin;
wherein the flavoring agent is an artificial meat or beef flavor.

5. The composition of claim 1, comprising:
a. a filler selected from corn starch, pre-gelatinized corn starch, corn gluten meal and soy protein fines, or a combination thereof;
b. a solvent selected from liquid polyethylene glycols, propylene glycol, propylene carbonate, caprylic/capric triglycerides, caprylic/capric/linoleic triglycerides, caprylic/capric/succinic triglycerides, propylene glycol dicaprylate/dicaprate, glycerol caprylate/caprate and polyglycolized glycerides, or a combination thereof;
c. a binder selected from polyvinylpyrrolidone, polyethylene glycols, co-polymers of vinyl acetate and vinylpyrrolidone, potato starch and corn starch, or a combination thereof;
d. a humectant selected from glycerol, propylene glycol, cetyl alcohol, glycerin monostearate and polyethylene glycols, or a combination thereof;
e. a surfactant selected from glyceryl monooleate, polyoxyethylene sorbitan fatty acid esters, sorbitan esters, polyvinyl alcohol, polysorbates, sodium lauryl sulfate, co-polymers of ethylene oxide and propylene oxide, propylene glycol monolaurate, glycerol caprylate/caprate, polyglycolized glycerides and polyethylene glycol hydroxystearate, or a combination thereof and
f. a natural or artificial beef or meat flavor;
wherein the composition comprises a IgY antibody at a concentration of about 1% to about 20% by weight.

6. The composition of claim 1, wherein the composition comprises a combination of at least one antimicrobial IgY antibody, active against oral health compromising (OHC) microorganisms, and at least one systemically-acting active agent selected from one or more arthropodicidal isoxazolines, one or more macrocyclic lactones, one or more spinosyn compounds, one or more spinosoid compounds, one or more benzimidazoles, levamisole, pyrantel, morantel, praziquantel, closantel, clorsulon, one or more amino acetonitrile active agents, one or more insect growth regulators, one or more neonicotinoids and one or more aryloazol-2-yl cyanoethylamino active agents, or a combination of thereof.

7. A method for the treatment and/or prevention of an oral health compromising (OHC) microorganism infection in an animal's oral cavity comprising administering to the animal an effective amount of the composition of claim 1.

8. The method of claim 7, wherein the OHC microorganism is S. mutans, S. salivarius, S. mutans, S. sanguinis, S. sobrinus, P. gulae, P. salivosa, P. gingivalis and P. denticanis.

9. The method of claim 7, which includes reducing the occurrence of dental caries or other oral health diseases or pathologies in an animal comprising the steps of:
a. selecting an animal with or at risk of developing oral health diseases or pathologies, including dental caries; and
b. contacting the oral cavity, and particularly the teeth, of the animal with a therapeutically effective amount of the antimicrobial IgY antibody-containing composition of claim 1; and
wherein the antibody is present in a safe and effective amount to reduce or eliminate adherence of one or more oral health compromising (OHC) microorganism(s) that cause oral or dental diseases or pathologies, thereby reducing the occurrence of dental caries or other oral health diseases or pathologies; and
wherein the IgY antibody is specific for at least one of the following OHC microorganisms: S. mutans, S. salivarius, S. mutans, S. sanguinis and S. sobrinus, P. gulae, P. salivosa, P. gingivalis and P. denticanis; and
wherein the therapeutically effective amount of the antimicrobial IgY antibody reduces the number or adherence of the OHC S. mutans, S. salivarius, S. mutans, S. sanguinis and S. sobrinus, P. gulae, P. salivosa, P. gingivalis and P. denticanis.

10. The method of claim 7, wherein the IgY antibody is an oral treatment form selected from a toothpaste, mouth rinse, gel, foam, varnish, polish, floss, dental tray, dental strip, copolymer membrane, and slow release bead;
wherein the IgY antibody is present in the oral supplement in a dosage of less than 1 mg/ml; or
wherein the IgY antibody is present in the oral supplement in a dosage of less than 100 μg/ml; or
wherein the IgY antibody is present in the oral supplement in a dosage of 50 μg/ml; or
wherein the IgY antibody is present in the oral supplement in a dosage of 25 μg/ml.

11. A composition, comprising:
a. a transfer agent;
b. a barrier material;
c. an antimicrobial IgY antibody, which is active in reducing or eliminating the numbers of one or more oral health compromising (OHC) microorganism from the oral cavity, including the teeth, of an animal;
wherein said transfer agent is present in an amount of 0.25 to 25 wt. %, based on the total weight of said transfer agent and said barrier material; and said barrier material is present in an amount of 75 to 99.75 wt. %, based on the total weight of said transfer agent and said barrier material; and
wherein said transfer agent is selected from lecithin, cetyl amine, N-tallow-1,3-propanediamine, hexetidine, and cetylpyridinium halide.

12. A method for producing antimicrobial IgY antibodies, which are effective in reducing or eliminating oral health compromising (OHC) microorganism, comprising the step of administering to a hen at least one OHC microorganism-associated polypeptide having a sequence having at least 60, 70, 80 or 90% identity to an immunologically sufficient portion of a sequence as set forth in any one of the following sequences: SEQ ID NO: 110-113, 120, 123-125, 130-133, 135-138, 143-148, 221-224, or 227-349.

13. A composition comprising one or more antimicrobial IgY of claim 11.

14. The composition of claim 13, wherein the composition is formulated as a gel.

15. The composition of claim 14, wherein the gel comprises CARBIGEN M at an optimal viscosity for mixture with the IgY and subsequent application to a canine's tooth.
